(19) United States
(12) Patent Application Publication
(10) Pub. No.: US 2002/0022240 A1
(43) Pub. Date: Feb. 21, 2002

(54) Method of assessing the biological status of cancer development

(76) Inventors: Gregory R. Wade, San Francisco, CA (US); Gabriel G. Altmann, London (CA); Stephen G. Altmann, Dundas (CA); Vilma Gejadharsingh Altmann, London (CA); Martine Altmann, Montreal (CA)

Correspondence Address: FAY, SHARPE, FAGAN, MINNICH & McKEE, 1100 Superior Ave., Seventh Floor, Cleveland, OH, 44114-2518, US

(21) Appl. No.: 09/783,477

(22) Filed: Feb. 14, 2001

(30) Foreign Application Priority Data
Feb. 17, 2000 (CA) 2,299,210

(51) Int. Cl. G01N 33/574; G01N 1/30; G01N 33/48

(52) U.S. Cl. 435/7.23; 435/40.5

(57) ABSTRACT

This invention provides a method for indicating a high or low risk of developing pathology at specific sites along epithelial tissues, according to a model of carcinogenesis and measurements of activated enterocytes. The method determines the presence and the intensity of a “promoting environment,” a region of intestinal epithelial cells which are biochemically programmed as activated enterocytes to develop pathology such as neoplasia or such as cancer in response to certain signals. The model predicts that neoplasia can develop only in such an environment when the promoting influence is sufficiently intense. In which case to provide the ability to identify pathologic tissues If cancer is determined to be present, this method enables one to assess the stage of cancer, which can be used to monitor the effectiveness of therapeutic regimes during the course of treatment for a patient. The method can also be used in the field of cancer research to develop understanding of the etiology of the disease in addition to new treatments for cancer therapy. It also provides an opportunity to use the method as a tool to investigate pathologic development such as pre-cancerous states in animal and cell culture models of diseases.

### FIELD OF THE INVENTION

This invention pertains to the field of cancer diagnosis, research and therapeutics.

### BACKGROUND OF THE INVENTION

Colorectal carcinoma is the second most frequent cause of cancer mortality in men and women, causing nearly one third of all malignancy-related deaths in North America. It has been estimated that ultimately as many as 6% of Canadians and Americans will develop malignancy in the lower bowel, and over 50% of them will die within 5 years of diagnosis. Because there are no realistic prospects of significantly improving the cure rate once the cancer has spread beyond the bowel wall, many authorities believe that colorectal cancer can be controlled only by preventive measures (Lieberman D. A, Amer. J. Gastroenterol. 1992, 87, 1085).

Primary prevention, i.e. averting the development of the tumour by altering biological risk factors, is not yet feasible since so little is understood of the etiology of the disease. Alternatively, secondary preventive measures, i.e. detection at an asymptomatic, treatable state, would be possible should an effective screening test be available. Indeed, neoplasms of the lower bowel have the characteristics that make them a suitable candidate for the development of a screening test: 1) because it is a common cause of cancer-related deaths; and 2) whereas once the stage of true cancer is reached, leading to symptoms, the mortality rate is over 50%, removal of bowel neoplasms at its earliest, asymptomatic stage can be done by non-surgical endoscopic polypectomy, without any significant risk. Moreover, it requires at least four to six years before an adenomatous polyp reaches the cancer stage, so there is ample opportunity to detect these neoplasms at their treatable stage.

Ulcerative colitis is a chronic, idiopathic inflammatory process of the colon which affects about 0.05% of the population of the Northern industrialized world. The disease is characterized by recurrent bouts of diarrhea and rectal bleeding that may require lifelong medical management. More importantly, it is now well recognized that ulcerative colitis is a premalignant condition, and it has been estimated. that about 13% of patients with pancolitis will develop carcinomas. About 1% of all new cases of colon cancer in this country arise as a complication of chronic inflammatory bowel disease. Compared to most colonic malignancies, these cancers tend to occur in a younger age group, to be multifocal, and to behave in a more aggressive fashion. It is believed that the majority of malignancies in ulcerative colitis can be prevented, because the epithelium of the affected colon undergoes premalignant dysplastic changes prior to the development or carcinoma and these premalignant changes can be detected by regular surveillance biopsies. Since the risk of malignancy increases with duration of disease, being about 5% at 15 years and increasing by 20% with each subsequent decade, yearly colonoscopy and surveillance biopsies are recommended for every patient with ulcerative colitis beginning at 7 to 10 years after diagnosis.

The problem that remains to-date, however, is that polyps can be reliably detected only by endoscopy. Six or seven random biopsies from different regions of the colon are usually taken at each colonoscopy. Typically, histological preparations are made of the biopsies, constituting thin sections that are examined under the microscope. The microscopic image of the colon is usually quite regular and predictable. In precancer and cancer, focal irregularities, so called lesions, may be found within the otherwise normal looking tissue. The pathologists then classify the lesion(s) found and accordingly, the degree of cancer development is determined.

Dysplasia cannot be recognized with the naked eye. Thus, directed biopsy is precluded, and sampling error is a major limitation to the efficacy of the procedure. The biopsy specimens themselves are, in turn, difficult for pathologists to interpret due to the atypical cytologic changes produced by acute inflammation or epithelial regeneration (healing) that resemble dysplasia. Interobserver variation in interpretation of surveillance biopsies varies by 4 to 8% among experienced pathologists and is undoubtedly much higher among non-expert pathologists. These problems will be greatly alleviated or even eliminated if dysplasia can be recognized both grossly and microscopically.

Screening tests are different from histological biopsies. The aforementioned endoscopic methods, such as sigmoidoscopy or entire-length colonoscopy, are diagnostic rather than screening techniques.

Recent clinical studies document a decrease in mortality from colorectal cancer screening.

Present techniques such as HemOccult II involve smearing a sample of stool onto guaiac impregnated paper which, after treatment with hydrogen peroxide containing developer, exhibits blue colour if blood, haemoglobin, is present. After almost two decades of experience with this methodology, it has become clear that even in expert centres, the sensitivity is less than 50% for curable neoplasms, and that the positive predictive value approximates, at best, only 40% in a clinic population. An update from the large-scale (n=97, 205) University of Minnesota, Minnesota, United States, prospective trial indicates a positive predictive value for colorectal cancer of only 2.2% when HemOccult is used in asymptomatic subjects, aged 50-80, with an overall disease prevalence of 0.2%. (Mandel J. S., Gastroenterol. 1989, 97, 597.) Furthermore, factors such as medications, multiple dietary constituents, delays in specimen handling, variabilities in faecal hydration, and storage of assay materials commonly confound results. Analysis of one of the three randomized controlled studies assessing the value of HemOccult suggests comparable mortality rates in the screened and control populations (Selby J. V.,.Ann. Intern. Med. 1993,118, 1.). Newer methods of detecting occult blood, e.g. methods based either on porphyrin analysis [HemoQuant] or antibody specific for human haemoglobin, may improve on these results.

However, three limiting problems remain unlikely to be overcome. These are that colorectal malignancies shed blood only intermittently, upper gastrointestinal tract bleeding may make the results (falsely) positive, and multiple lesions in the lower bowel, apart from colorectal neoplasms, commonly bleed. Such lesions include haemorrhoids, diverticulae, ulcers, and vascular ectasie. Compliance in unselected populations has been estimated to be less than 30%, at least partly because the technique requires patients themselves to smear their stool onto a slide or a strip, a task most people find not only distasteful, but also technically difficult. Despite this, HemOccult continues to be widely used because the American Cancer Society has recommended occult blood testing yearly for all individuals over 50 years of age, arguing that even an imperfect test will save many lives. Implicit in all arguments over the value of HemOccult is that any improvement in screening techniques for bowel malignancy would have a dramatic impact on colorectal cancer mortality rates from the disease, since the screening for occult blood even in the present form leads to reducing mortality from colorectal cancer (Mandel J. S.,New Engl. J. Med. 1993, 328, 1365).

Screening for colorectal cancer by stool DNA analysis (Lancet 1992, 339, 1141) is based on the presence in stool of neoplastic cells shed in large numbers into the colonic lumen. In principle, a mutation which is common to neoplasms could be detected with high precision by analysing DNA from these cells. Therefore, the existence of a detectable mutation in the colorectal tumour is a prerequisite for developing such a method of screening. Unfortunately, this technique can recognize a mutation based only on a new or altered oligonucleotide sequence, but not on a loss of its portion. Thus, neoplasia-related mutations based on deletion in genes, e.g. allele losses on chromosomes such as are commonly found in colorectal tumours, are beyond the limits of the methodology. Currently, the most common mutation is the K-ras oncogene mutation present, in about 40% of colorectal carcinomas and adenomas. Screening for K-ras gene can therefore detect, at best, only 40% of all neoplasias. This methodology is at present technically very complex and expensive.

Screening for the presence in colonic mucin of a cancer-related disaccharide, D-Galp(β1-3)-D-GalpNAc(αl,Ser/Thr), T-(Thomsen-Friedenreich) antigen, since it is widely known that T-antigen is not expressed by cells in healthy colons, whereas it is expressed by cancer (Boland C. R., Proc. Natl. Acad. Sci. USA 1982, 79, 2051).

Monoclonal antibodies and lectins:

It has been shown that monoclonal antibodies raised against synthetic T-antigen recognize and bind to cancer cells. Similarly, peanut agglutinin (PNA), a lectin, binds strongly to the same disaccharide, but recognizes malignancy with lesser specificity. Amaranthin, a lectin from Amaranthus caudatus, has been reported to have better specificity for T-antigen than PNA. Neither amaranthin nor PNA bind to histological sections of normal mucosa, but both bind to mucin in the goblet cells of tumours and certain polyps, and in the transitional mucosa. The visualization of the binding utilizes fluorescently labelled antibodies and lectins (Rinderle S. J., J. Biol. Chem. 1989, 264, 16123.).

Galactose oxidase test.

T-antigen is also reported to be detectable colorimetrically after oxidation of OH-6 of galactose using galactose oxidase and visualization of the resulting aldehyde with Schiff's reagent (U.S. Pat. No. 4,857,457, issued Aug. 15, 1989 to Shamsuddin et al.). In contrast with the tests using lectins, this test is performed on mucus samples obtained by digital rectal examination and smeared onto a support. This system demonstrated a sensitivity of 74% and specificity of 50% for colorectal neoplasms, i.e. adenomatous polyps and cancer, in one study with only 1 false negative result among 59 patients with cancer. Since then a number of reports of basically the same test have appeared with sensitivity ranging from 35% to 100% and specificity ranging from 15% to 76%. Some investigators found that the test was more sensitive, but less specific, than HemOccult. The lesser specificity has been ascribed to the positivity of test in individuals with certain inflammatory condition, such as diverticulitis and ulcerative colitis (Sakamoto K., Cancer Biotherapy 1993, 8, 49).

Prior art methods such as that taught in U.S. Pat. No. 5,416,025 teach methods for detecting the presence of cancer of the colon or rectum by treating a sample of colorectal mucus from the rectum of a patient with Schiff's reagent, wherein colouration produced in the sample indicates the presence of cancer. This method, however, can only indicate the presence of cancer somewhere in the colon without indicating the source because the mucus from various sites tend to accumulate in the rectum. The results of this test are likely influenced by bowl habits, the way mucus is collected, drugs taken, etc. Most importantly however, the results are not proportional to the degree of cancer and will only pass a threshold reading of positivity once the cancer reaches a sufficiently advanced stage of development, that the marker substance is released into the mucus. Moreover, the area of the cancerous colon that is responsible for releasing the Schiff positive marker substance is unknown.

The current diagnostic and screening methods entail detecting the presence or absence of a marker that collects in the mucus, stool or blood. These methods usually are limited to provide a qualitative positive or negative reading, which can be significantly affected by other factors present in the colon. Moreover, these methods fail to provide predictive information and they do not provide understanding of the etiology of the disease.

### BRIEF DESCRIPTION OF THE DRAWINGS

Table 1 presents the Plasmalogen Indices Recorded for each case. The results from histometric measurements on individual samples from various groups are listed, where an index of 10 to 30% plasmalogen index is considered to be within a normal range, an index of 30 and 40% is considered to be moderately elevated and an index above 40% is considered to be high.

FIG. 1 depicts the basic structure of the intestinal epithelium. The level between the intestinal lumen and the tissue surface is indicated by the horizontal heavy lines. In the small intestine, the epithelium is thrown into finger like projections, the villi. Villi are not present in the colon. In both, small intestine and colon, the epithelium deeps down into the tissue to form the cylindrical structures, the crypts. The epithelium is simple columnar, it is continuous so that it covers the luminal surface of the tissue including the villi and it lines the crypts. The main cell type of the epithelium is the enterocyte that is the absorptive cell (E). The mucus-producing goblet cells (G) are scattered singly within the epithelium. Fibrous connective tissue (F) fills up the space in the core of the villi and between the crypts. The whole epithelium is in a continuous movement as new cells are being added to it in the lower half of the crypts by the cells dividing there (M). The newly added cells are mainly immature enterocytes which move upwards to the crypt and cease dividing after about 3 transit divisions. They then reach the upper crypt, the compartment of maturing cells. They continue migrating and become mature by the time they reach the villus or the surface epithelium of the colon. They then continue migrating and function at the same time.

They are lost by exfoliation after reaching the villus tip or the colonic surface epithelium at midpoint between adjacent crypt openings. The crypt base is organized differently: slender stem cells are located between large “nurse” cells which are the Paneth cells in the small intestine and the deep crypt secretory (DCS) cells (Altmann, 1990) in the colon. The stem and nurse cells do not participate in the upward migration of cells.

FIG. 2 illustrates details of a crypt. The cells are localized in well distinguished compartments.

FIG. 3 shows the basic concept of the renewal of the intestinal epithelium. The stem cells orchestrate the renewal as their divisions produce the early transit cells which then enter the transit compartment. Stem cell divisions also provide for new stem cells so that the stem cell compartment is maintained for life. The transit cell divisions provide for daughter cells that are more mature than the parental cells. After such maturation and divisions, the daughter cells reach a nonproliferative stage and enter the maturing compartment where the maturation toward functional end cells continues.

FIG. 4 illustrates the concept of precancerous hyperplasia. The primary change is in the stem cell compartment which is enlarged by the addition of initiated stem (IS) cells. These then produce their progeny of initiated cells which renew similarly to the normal enterocytes (compare with FIG. 3). They thus go through the transit, maturing and mature compartments and then exfoliate. The initiated progeny coexists with the normal enterocyte population which is not affected by the carcinogen. One can visualize that if the IS cells enter the dormant nonproliferative Go stage, the initiated progeny will exfoliate and hyperplasia will regress. After subsequent activation of the IS cells, they will reconstitute the initiated progeny.

FIG. 5 illustrates the genesis of tumours. I. Normal crypt base. Paneth and stem cells alternate in a regular manner. II. Irregular crypt base. An altered focus appears from which one or few basophilic small cancer cells arise. III. Early cancer cell accumulation. IV. The cancer cells spread from the focus and fill most of the crypt base. V. Carcinoma in situ. The accumulation takes up the shape of a tumour. At the same time, streams of cells proceed toward nearby blood or lymphatic vessels. VI. Metastasis. Some cancer cells reaching the vessels develop secondary tumours which eventually disseminate via the circulation.

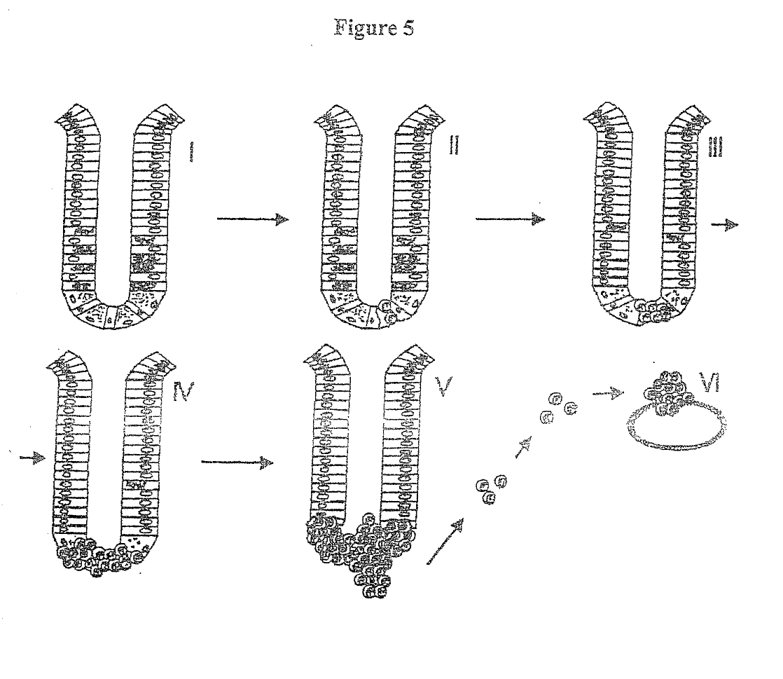

FIG. 6 shows a generalized illustration of our conclusions on cancer development. Primarily, the stem cells are affected by various environmental agents and they go through various stages until finally cancer (neoplastic) stem cells arise. Each type of stem cells produce a different cell type and a different lesion type. There are 2 major categories of preneoplastic (precancerous) cell types as shown. The preneoplastic and the neoplastic stem cells are inhibited by NK cells from producing their progeny.

FIG. 7 shows the life cycle of the colonic enterocytes. From the stem cell stage, they proceed to deep crypt secretory cells which accumulate glycoprotein containing secretory granules (DCS or vacuolated cell). They soon degranulate and transform into enterocytes, one of the functions of which is phospholipid production, mainly in the apical cytoplasm, the rate of which is apparently determined by environmental influences. Since plasmalogens are phospholipids, the histological procedure should not use lipid solvents. The goblet cell line is independent but it is difficult to distinguish goblet from DCS cells in the deep crypt region. In midcrypt and above, the DCS cells lose granularity whereas the goblet cells retain it.

FIG. 8 presents a drawing illustrating well developed enterocytes containing large amounts of plasmalogen in their apex. This figure shows that the phospholipid formation is exaggerated in areas where promoters are accumulating. Here the probability of cancer arising is high. The morphological signs of these areas include (i) enterocytes distended with lipid (lipo-, or I-enterocytes) (ii) apical bands caused by accumulated I-enterocytes, and (iii) elevations caused by highly active accumulated I-enterocytes. We have called these cells also lipo-, or 1-enterocytes as the plasmalogen content is lipid-like. The presence of these cells mark out cancer prone areas. Together they provide for a “band” or a “film” over the colonic surface. Some parts of these bands also appear as elevations.

FIG. 9 presents PLATE 1, comprising frozen sections from the human colon biopsies stained with Schiff after mordanting the sections with mercuric chloride. There is a general pink tissue staining whereas the plasmalogen positive areas display strong magenta colour, with typical apical bands an delevations. There was a patchy distribution of positive areas but their frequency was high in the high cancer risk colon. The three pictures on the left, display largely negative surface epithelium which, however, still contains about 10-20% plasmalogen. The pictures on the right, display highly positive surface epithelium with 40-60% plasmalogen. Plasmalogen bands and elevations are clearly visible. All ×75.

FIG. 10 shows mouse ascending colon, fixed in Carnoy solution, embedded in Historesin, simithin section, iron-hematoxylin (IH) stain. This region of the mouse colon shows clearly the three main regions of the epithelium: deep crypt secretory (DCS) (lower arrow), intermediate, and surface epithelial (upper arrow). In the DCS region, the large secretory cells (lower arrow) filled with granules are most prominent. Occasionally a small group of narrow columnar cells without granules can also be made out near the crypt bottom (not shown here); these are the stem cells. According to the histological evidence, also shown earlier (Altmann, 1990), the immediate derivatives of the stem cells fill up with granules probably of glycoprotein nature. Such cells with relatively few granules have been referred to traditionally as vacuolated cells. In some areas, as here for example, the granules are abundant, in which case we refer to the cells as DCS cells. In vacuolated as well as DCS cells, most granules eventually exocytose. This activity is very prominent with the DCS cells. The lower third of crypt lumen is usually seen to fill up with the granules. After this exocytosis, the cells get into the intermediate zone as columnar or transit cells, increase in size gradually and reach the surface as surface enterocytes (upper arrow). ×900.

FIG. 11 shows mouse ascending colon fixed in osmium-permanganate. Semithin section stained with IH. Almost through the entire lower crypt, DCS cells are seen to be releasing granules en mass into the crypt lumen. Few cells still full of granules are seen to reach the midcrypt in the upper part of the picture. These cells are goblet cells which remain filled with granules until their exfoliation near or at the luminal surface of the colon. ×1000.

FIG. 12 shows the same tissue as in FIG. 11 but in the midcrypt position. The several cells retaining a few DCS granules are transit cells. The few that still retain full granularity are goblet cells. The goblet cells retain their morphology after deriving from stem cells; they form thereby a lineage separate from that of the DCS-enterocyte line. ×1000.

FIG. 13 shows mouse colon frozen section stained for plasmalogen. The most positive reaction is in the surface enterocytes. The reactive area seen as black was magenta in the original section. ×450.

FIG. 14 shows human colon, frozen section, Schiff plasmalogen staining. Part of the surface epithelium is shown from a “cancer prone” area. Most plasmalogen is in the apical portion of enterocytes where it may form “bulbous” elevations (B). Many of these cells also contain remnants of DCS cell granules (D), which did not exocytose with the rest. ×950.

FIG. 15 shows human colon, frozen section, Schiff plasmalogen staining but now counterstained by hematoxylin to improve visible cellular detail. Clear-cut 1-enterocytes can be observed in this picture. Their apex is filled with the lipid-like plasmalogen. These apices face the colonic lumen (Lu). Because of this cellular plasmalogen accumulation, these is a plasmalogen “band” or “film” over the colonic surface except where goblet cells (G) are present. ×1020.

FIG. 16 shows human colon fixed in Carnoy solution. Semithin section stained with IH. Large well developed DCS cells can be seen in lower crypt. Most reach the crypt lumen, some are passing several secretory granules into this lumen. In the upper crypt, the granularity of the individual DCS cells is much reduced as those cells become transit cells. ×1220.

### SUMMARY OF THE INVENTION

The method of this invention provides a novel approach to diagnosing and characterizing the stage of cancer, which also assists in designing and monitoring the course of therapy. This approach uses a histopathological biomarker, the activated enterocyte (I-enterocyte), to accurately characterize the status of cancer development with more accuracy and efficiency than standard techniques, and can even enable the determination of the potential of cancer to develop in response to carcinogens.

Another object of the present invention is to provide a diagnostic test for colorectal carcinoma which detects a biochemical change, such as the accumulation of plasmalogen in activated enterocytes, that is associated with the stage of cancer development. According to Webster Third New International Dictionary, plasmalogen is a phosphatide that is the precursor of plasmal in tissue.

A further object of the present invention is to provide a kit by means of which such tests can be conducted to determine the stage of cancer development.

It is a further object of this invention to provide a method to determine the effects of different therapeutic regimens that will enable one to design the most effective course of treatment for a patient.

In a further embodiment, the method of this invention provides a means for studying the underlying cause/changes that lead to cancer.

In yet another embodiment, the method of this invention provides a means to identify pre-cancer point or individuals with likely propensity to develop cancer.

In yet a further embodiment, this invention provides an intermediate endpoint which can be used as an indicator of health and the likelihood of developing of cancer and the effect of a prophylactic therapy.

Another embodiment of this invention can be used to develop animal models in which the effects of agents, genes, environmental factors, can be researched via their effects on the intermediate endpoint.

Upon study of the specification and appended claims, further objects, features and advantages of the present invention will become more fully apparent to those skilled in the art to which this invention pertains.

### DETAILED DESCRIPTION OF THE INVENTION

The first object of the present invention is a new and efficient method of determining whether or not an animal or a human is at risk of developing a pathology such as a cancer pathology and more particularly a colorectal cancer pathology. This method comprises the steps of:

(i) obtaining a sample from an animal or from a human source, which sample is preferably selected in the group constituted by samples of intestinal tissue, mucus samples and samples of washings collected from the colon;

(ii) determining if said sample comprises activated I-enterocytes; and

(iii) concluding if the animal or human is at risk of developing colorectal cancer.

A second object of the present invention is a method of determining whether or not an individual is at risk of developing a colorectal cancer comprising the steps of:

(i) obtaining a sample from the individual;

(ii) determining if said sample comprises activated I-enterocytes; and

(iii) concluding if the individual is at risk of developing colorectal cancer.

According to a preferred embodiment of the invention, in step (i), the sample is a sample of epithelial tissue such as samples of intestinal tissues, mucus samples and samples of washings collected from the colon. The samples are preferably recovered by biopsy, by surgical excision or by mucosal scrapping.

According to a further preferred embodiment, in step (ii), the activated I-enterocytes is selected in the group constituted by I-enterocytes with altered morphology. This altered I-enterocytes may be I-enterocytes with aprical abnormalities such as I-enterocytes with exaggerated phospholipid formation in areas wherein promoters of cancer pathology, preferably in areas wherein cancer promoters are accumulating.

According to a preferential embodiment of the invention, the intestinal tissue which is recovered in step (ii) by biopsy is embedded into paraffin or plastic blocks and then cut into thick sections, preferably cut into about 5 micrometer thick sections.

According to another preferred embodiment the thick sections recovered in step (ii) are mounted on glass slides, stained by immersing them into specific staining solutions and then examined under the microscope for the presence of activated I-enterocytes.

The thick sections are advantageously prepared and treated by mild acid hydrolysis or by dilute solutions of mercuric chloride before characterisation with a Schiff's reagent or with a modified Schiff's reagent in order to obtain an activated I-enterocytes biomarker.

A preferential modified Schiff's reagent is obtained from a traditional fuchsin solution by the following 15 steps process:

1. Filtering the fuchsin solution through pleated filter paper and collect in a 1000 mL Erlenmyer flask

2. Measuring 68 mL of IN Hydrochloric acid. Record actual quantity above.

3. Adding 1 N hydrochloric acid to the solution while stirring. Allowing to dissolve completely. Letting solution cool to room temperature. Recording temperature.

4. Weighting out 4.68 g of sodium bisulfite. Recording actual quantity above.

5. Adding the sodium bisulfite while stirring. Allow to dissolve completely.

6. Covering the Erlenmyer flask with parafilm and store in a dark cupboard at room temperature for 4 days.

7. Confirming colour of solution, light straw colour.

8. Weighting out 0.60 g of decolorizing charcoal. Recording actual quantity above.

9. Adding the charcoal to the solution. Stir for 1-2 minutes.

10. Filtering solution into a 500 mL graduated cylinder through two Whatman filters. The bottom filter is a disc and top filter is pleated. Stir as required.

11. Measuring volume. Recording the volume.

12. Measuring the pH, (it should be approximately 1.1). Recording the pH.

13. Transfering solution into 500 mL brown glass bottle. Close bottle with screw cap. Label with name (Schiff Reagent), date, and lot number.

14. Labelling bottle as “Quarantined” and storing the bottle in a refrigerator at 2-6° C.

15. Thirty days after production is completed, taking of samples and performing of QC release tests.

The corresponding modified Schiff's reagent thereby obtained, may still be generally qualified as a Schiff's reagent which is characterised by an enhanced storage stability and colour development ability.

According to the present method, it is possible in step (iii) to conclude that the animal or human is classified at risk of developing a pathology if a deep magenta colouring may be detected in any part of the sample prepared in step (ii) and to conclude that the individual is classified at no risk of developing cancer if no deep magenta may be detected in the sample prepared in step (ii).

A third object of the present invention is constituted by a new method of determining the stage a pathology in an animal or human with a pathology such as neoplasia or such as a cancer pathology(for example a cancer pathology). This method comprises the steps of:

(i) obtaining a sample from the animal or from the human, which sample is selected in the group constituted by samples of intestinal tissue, mucus samples and samples of washings collected from the colon;

(ii) determining if said sample comprises elevated levels of activated I-enterocytes; and

(iii) quantifying the elevated level of activated I-enterocytes and correlating said elevated level of activated I-enterocytes to the stage of the pathology.

According to a preferred embodiment, the sample of intestinal tissue is, after staining by a Schiff's reagent or after staining by a modified Schiff's reagent, evaluated for its level of activated I-enterocytes which is proportional to the percentage area of the sample showing a deep magenta colouring.

According to another preferred embodiment, the level of activated I-enterocytes is qualified of high if more than 30% of the area of the sample shows a deep magenta colouring.

A fourth object of the present invention is constituted by a new method of determining the localisation and the stage of a pathology in an animal or human:

(i) obtaining samples from different and identified parts of the animal or human, which samples are selected in the group constituted by samples of epithelial tissues such as intestinal tissues, mucus samples and samples of washings collected from the colon;

(ii) determining which of the samples collected in step (i) comprise activated I-enterocytes;

(iii) quantifying the level of activated I-enterocytes present in the samples identified in step (ii) as comprising activated I-enterocytes; and

(iv) correlating the results obtained in step (iii) to the localisation and to the stage of the pathology.

A fifth object of the present invention is a new method of determining the risk for a patient to develop a pathology such as a cancer pathology (for example neoplasia or colorectal cancer). This method comprises the steps of:

(i) obtaining a sample of an epithelial tissue from the animal or from the human;

(ii) determining if said sample comprises elevated levels of plasmalogen in the intestinal epithelial tissue;

(iii) correlating said elevated level of plasmalogen to observations made in normal non-initiated tissue; and

(iv) quantifying the stage of pathology if the individual has developed pathology.

A sixth object of the present invention is a kit for working one of the five above defined method of diagnostic. This kit comprises:

(i) at least one reagent forvisualising the presence of plasmalogen (preferably the presence of I-enterocytes) in the sample(s); and

(ii) a reagent for identifying the presence of activated I-enterocytes in the sample(s).

According to a preferred embodiment of the kit, the reagent for visualising the presence activated I-enterocytes in the sample(s) is a mild acid or dilute mercuric chloride.

A seventh object of the present invention is a kit for working any of the above defined diagnostic methods. This kit comprises at least one reagent for oxydizing said tissue; and a Schiffs reagent or a modified Schiff's reagent.

The reagent for oxidising said tissue is preferably selected in the group constituted by inorganic acids, mercuric chlorides and solutions (preferably aqueous) solutions thereof. This kit may be advantageously completed by a third component which is instructions for working the diagnostic method.

A eight object of the present invention is the use of the above defined methods of the invention as a tool to investigate pathologic development such as pre-cancerous states in animal and cell culture models of disease.

The method of this invention provides a novel approach to diagnosing and characterizing the stage of cancer, which also assists in designing and monitoring the course of therapy. This approach uses a histopathological biomarker, the activated enterocyte (I-enterocyte) in the intestinal epithelium, to characterize the status of cancer development with more accuracy and efficiency than standard techniques, and can even enable the determination of the potential of cancer to develop in response to carcinogens. This biomarker derives from the following model of carcinogenesis.

According to this model, carcinogens do not cause cancer directly. Rather, they establish a permanent set of enterocytes in the intestinal epithelium, that is slightly different from the normal ones but being still functional as well as nonmalignant. These cells are called initiated cells. Their presence causes no ill effects. However, these cells can respond to another set of environmental substances, the promoters, by becoming cells responsive to mutagens. Some of these in the presence of mutagens may transform into typical cancer cells. While initiation does not necessarily lead to cancer, cancer development necessarily involves the prior sequence of initiation, promotion, and then mutagenesis. The method of this invention uses various techniques to determine the presence of activated I-enterocytes which enables one skilled in the art to define the status of cancer development with more accuracy and efficiency, in addition to providing a multifaceted understanding of the progression of the disease in accordance with this model, which is explained in further detail below.

The method of the invention derives from the following model of cancer development, which is a unified view of the sequence of events from initiation and latency period to metastatic lesions development (Altmann, G., Epith. Cell Biol., 1995). The histopathological marker enables the method of this invention is a method of staining the cellular tissue to evidence the activated I-enterocytes, primarily by determining the elevated levels of plasmalogen contained within the enterocytes, indicating a cellular status representing stages of cancer development that are relative to this model.

In particular, this invention provides a method for indicating a high or low risk of developing cancer at specific sites along the colon. The method measures the presence and the intensity of a “promoting environment,” a region of intestinal epithelial cells which are biochemically programed as activated enterocytes to develop cancer in response to certain signals. The model of this invention predicts that cancer can develop only in such an environment when the promoting influence is sufficiently intense.

At present, there are no clinical methods of testing for the presence of this environment. The method of this invention, provides a means for determining the presence of this type of environment by visualizing the activated enterocytes of intestinal epithelial tissue and correlating the signal measured to the propensity to develop cancer.

There are two important results obtainable from this kind of in situ test; 1) the degree of staining is proportional to the propensity to develop cancer, so this method can be used both qualitatively and quantitatively; and 2) the distribution of positivity is not uniform along the epithelium, so this method can be used to indicate regions of the colon at risk to develop cancer. Thus, this method can be conducted in vitro, analysing histological samples obtained from routine biopsies.

In one embodiment, the method entails treating a frozen or chemically fixed tissue biopsy slice with a solution that enables visualization of plasmalogens in the tissue, by either mild acid hydrolysis or by treatment with a dilute solution of mercuric chloride to liberate aldehydes which are then reacted with a Schiff reagent. One skilled in the art would appreciate the many means possible for first oxidizing the cellular material prior to reacting it with a Schiff reagent. This method can be used both qualitatively and quantitatively.

A Model of Carcinogenesis

Carcinogenesis, or cancer development, involves the essential stages of initiation, promotion, and progression. The basis of initiation seems to be the establishment of initiated stem cells which appears to be a high probability event involving most if not all crypts along the entire target organ of DMH, the whole intestinal tract. A genetic factor seems to be involved apparently regarding the nature of the stem cells, whether they are responsive or not to a carcinogen.

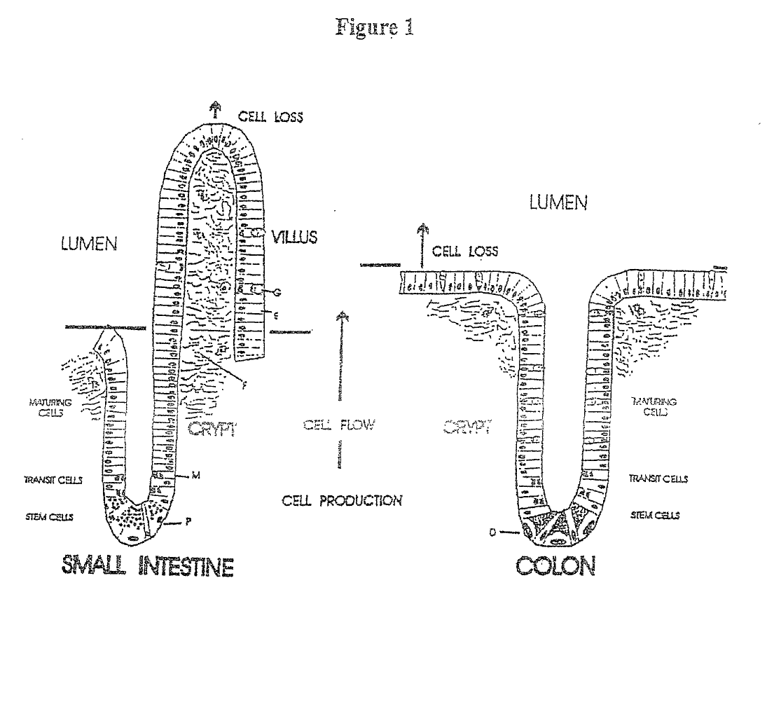

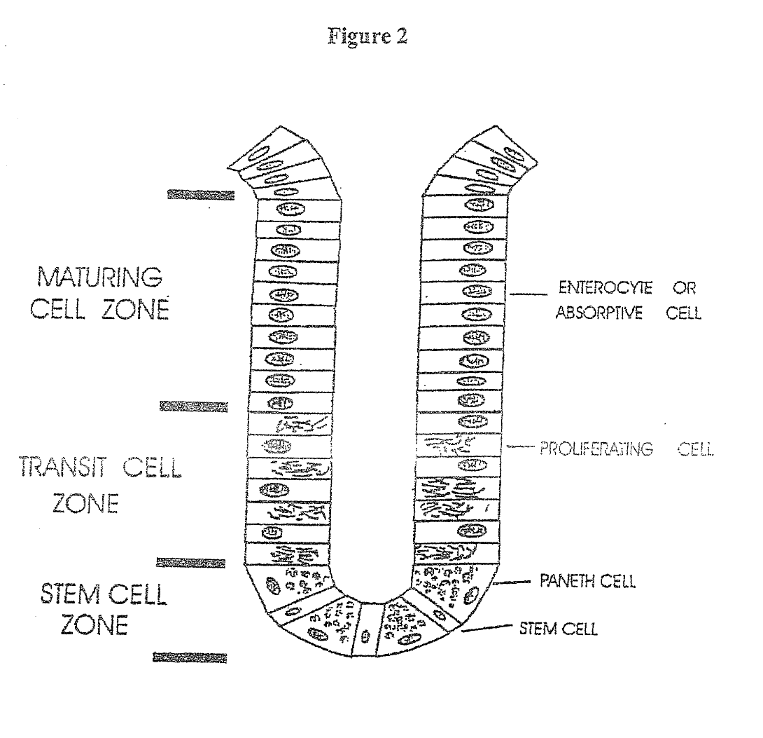

Stem cells are proliferative “transit” or “progenitor” cells which further differentiate as well as divide, then become nonproliferative maturing cells. These then mature into functional “end” cells. Although stem cells represent a small percentage (0.1-0.01%) of a renewing cell population, they are essential for the maintenance of this population as they renew themselves as well as produce the earliest cell types that become the renewing cell population itself. In the intestinal epithelium, the stem cells are at the bottom of the crypts and the transit cells occupy about the lower half of the crypts (FIGS. 1 and 2). The transit cells mature as they divide and after a set number of divisions, they reach a stage when divisions cease but maturation continues.

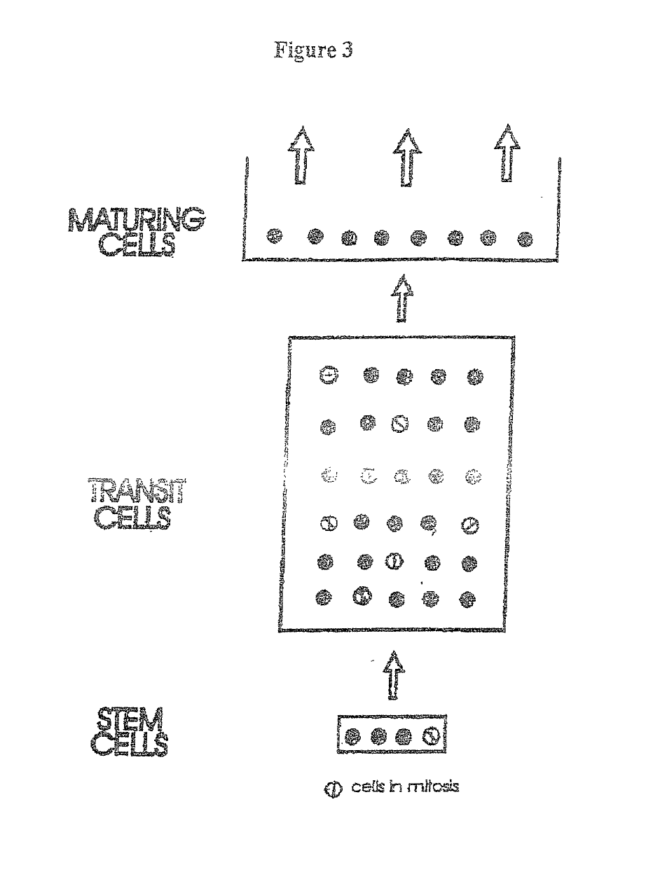

These nonproliferative maturing cells occupy the upper half of the crypts. By the time they reach the surface epithelium, they become mature “end” cells which exfoliate after functioning for 3-5 days. There are thus at least four compartments, stem, transit, maturing, and mature (functional) in a renewing cell population (FIG. 3). The same tissue may have more than one renewing system.

The stem cells are the ultimate source of renewal as their proliferation results in the self-renewal of the stem cell population itself as well as in the production of the earliest transit cells. These transit cells divide about three times, each division resulting in a pair of daughter cells more differentiated than the parent cell. Transit cells therefore cannot maintain their own compartment, rather their renewal is provided by the output of early transit cells from the stem cell compartment. If the divisions of the stem cells cease for any reason, that is the stem cell compartment is inactivated, the transit cell population eventually depletes, renewal stops and epithelial denudation follows as a result.

Stem cell function may naturally cease by the stem cells entering a resting or dormant phase. This is prevalent in slowly renewing tissues such as liver or muscle where the functional cells are long lived and need to be replaced mostly in case of injury, at which time, the dormant stem cells are reactivated. In the case of rapidly renewing cell populations, such as the intestinal epithelium, most stem cells are believed to be active. External influences seem to determine whether stem cells are in the active or in the dormant state.

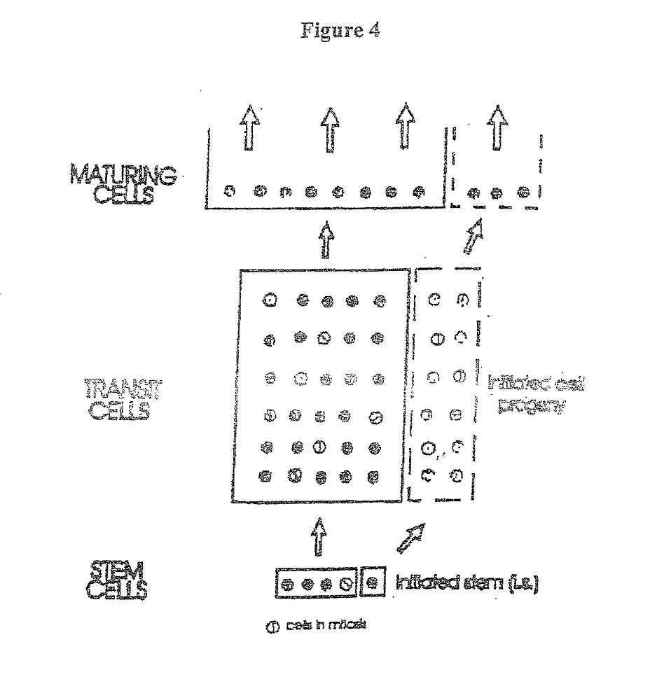

Hyperplasias are one of the earliest detectable lesions of precancer. There are two aspects of hyperplasia sometimes evaluated separately: 1) expansion of the cell content of a particular cell population of the tissue; and 2) an increase in the proliferation rate, where the surge of proliferation has been considered to be a prerequisite of cancerous transformation. In the current model, hyperplasia is caused by the appearance of additional cells in the renewing population and the pattern of renewal of these new cells is similar to normal. Consequently, aside from the overall enlargement of the tissue, there is no change in the relative content of the component cell populations and there is no disturbance in regular tissue architecture. The appearance of such added cells requires that the transit cells go through more than their set number of divisions or alternatively, new active stem cells make their appearance. In fact, there are new stem cells, “initiated stem” (IS) cells associated with the new population. They apparently arise from proliferation of normal stem cells which add the additional mitosis. Hyperplasia is thus visualized as an increase in all compartments of renewal, each compartment having an additional “initiated” component deriving from the newly added IS cells (FIG. 4). These cells are thus present all along the small and large intestines and follow the signals for renewal, that is, they migrate, differentiate and exfoliate. It is also important to note that they coexist with normal enterocytes, and that it is possible to measure the number of initiated cells by the degree of hyperplasia.

The natural killer (NK) cells are unique lymphocyte-like cells of the immune system, that have the ability to kill cancer and virus-infected cells. They react against not only cancer cells but also against the still noncancerous initiated and promoted cells. Under normal physiological conditions, the NK cells render all the three aberrant stem cell types nonproliferative. This state of the stem cells is referred to as “dormant” or “latent”. The NK cells secrete a paracrine factor which can bring all three populations, precursor I and II and cancerous, into remission so that only the stem cells survive in a harmless nonproliferative form. This factor is referred to as the remission factor. In the active state, the NK cells kill the initiated and the other aberrant cell types including their stem cells. NK cells are routinely activated by the cytokines interferon gamma or interleukin 2.

Hyperplasia is the lesion found to represent initiation by being composed of initiated enterocytes, which are the progeny of IS cells. A major control mechanism regulating the presence of this progeny comes from the NK cells which, if not suppressed, cause the dormancy of IS cells. Such dormant IS cells are the characteristic but elusive structures present in the otherwise normal tissue during the latency period ensuing initiation. They are responsible for the “memory” of the initial carcinogenic insult but they are not detectable by existing pathological tests. It appears that individuals may accumulate such dormant IS cells during their life span, that is being in the initiated state, with no ill effects.

While the IS cells constitute the basis for further progression, only a few may be involved, most remaining in dormancy. A pivotal local event in stimulating dormant IS cells appeared to be promotion. Promotion may be defined as a local mitotic stimulus on the IS cells. Under this stimulus, some mutagen-sensitive stem cells may arise, the PS cells, which may also give rise to local precancerous lesions. The PS cells may be much less frequent than the IS cells but still of high enough frequency which typical mutation could not produce. Promoters act by activating the proliferation of dormant IS cells, and this activation results in a nonmalignant transformation of some. It appears that this transformation may not be a mutation but rather a switch in stem cell regulation.

The progression stage involves the neoplastic transformation which can be elicited rapidly in initiated animals. The initiated state, previous promotion, mutagens, and NK inhibition are necessary for this to occur. Apparently, IS cells sensitized by promoters, that is PS cells, transform into NS cells. This time a very few cells are involved and true mutational events are probable. The connecting link between these lesions is their stem cells which can exist at least at three main levels of transformation. Whether or not these three stem cell types remain dormant or are activated to form lesions is dependent on environmental factors including a factor from the NK cells. A strong influence from the NK cells is capable of keeping most if not all of the transformed stem cells in the dormant state. Three basic stem cell types may be involved and all are under a strong control derived from the NK cells.

The renewal of the colonic epithelium is known to start in the deep crypt from stem cells which produce still proliferative transit cells. These gradually develop into non-proliferative surface absorptive cells which exfoliate after functioning for 4-5 days. The first transit cells arise from stem cells accumulate glycoprotein granules which then gradually release into the crypt lumen.

This process is referred to as deep crypt secretion, and the cells accomplishing this task have been named deep crypt secretory cells (DCS).

DCS, transit, and surface epithelial cells make up the epithelial continuum and represent the three major phenotypes of epithelial cells. The goblet cells arise separately from stem cells and continue to migrate as such to the surface. These cells produce large quantities of phospholipids.

This production is increased in the promoted state as shown after feeding promoter to the animals. The mechanism appears to be as follows: under the influence of the promoter, a large number of DCS cells form but they soon degranulate and thus become transit and then surface cells. The extent of the phospholipid producing epithelium is much increased.

Positivity is shown among some stem and progenitor cells in the crypt and as these cells approach the surface, they accumulate more plasmalogens. Some cells in high positive cases clearly overproduce this substance. This model predicts that at some stage of an enterocyte life cycle, large cytoplasmic areas switch from glycoprotein production to lipid production and this process is enhanced in cancer prone individuals. This is referred to as the “lipid switch”.

In this model, cancer does not arise directly from the normal cells. Rather, there are at least two groups of precursor cell populations: precursor cell type I or “initiated”, and precursor cell type II or “promoted”. The initiated cells arise after brief exposure to carcinogen, in its target organ. They have minor alterations but are still functional; they are additional to normal type cells, mingled with them adding 10-20% to the cell number comprised by the normal cells. Thereby, they cause an increase in tissue dimensions (hyperplasia) under repeated exposures to high concentrations of promoters, some initiated cells transform into promoted cells which form structures that are still nonmalignant but posses various degrees of typia. Few promoted cells may transform into cancer cells upon the influence of mutagens. There is thus a line of transformation from normal cells to initiated, promoted and then cancerous ones under environmental influences. Transformations take place primarily at the level of stem cells. Only stem cells can populate or repopulate cell populations. There are thus three groups of aberrant stem cell types: initiated (type I), promoted (type II), and neoplastic or cancerous (type III). They produce the following populations of enterocytes, respectively: hyperplastic (i.e. additional to normal), atypic (premalignant), and neoplastic (malignant or cancerous).

The nature of the latent period precedes cancer. Essentially, the latent period is the presence of aberrant stem cells in their latent form while no obvious lesions can be demonstrated. Stem cells in general are few (about one among 1000-10000 functional cells) and inconspicuous so that the latent period appears to be fully normal, the presence of a few aberrant stem cells cannot be demonstrated by routine pathological methods. Stage I of the latent period is when type I stem cells are only present. Stage II refers to the situation when type II stem cells are also present and stage III refers to the presence of latent cancer (type III) stem cells. Stages II and III are actually remission states as the stem cells sources of premalignant and/or malignant lesions are kept at bay. only when this rather effective immune control is breached at some location, cancer, which is a localized lesion, may develop.

In contrast to known diagnostic and screening techniques wherein the manifestation of cancer is observed from the perspective of the neoplastic growth, the method of this invention, diagnoses cancer from the point of view of the source of the neoplastic growth according to the model presented herein. Accordingly, the method of this invention uses biomarkers associated with stem cell progeny to analyse mammalian tissue samples for indications of the biological health status of stem cells. A key aspect of this invention draws upon the relationship between stem cells and their progeny vis-a-vis carcinogenesis; the aberrant stem cells give rise to foci of neoplastic growth, which provides biomarkers that can be detected as an indicator of aberrant stem cells.

Due to its focus on the source of neoplastic growth, the method of this invention can also be used to provide a means for studying the underlying cause/changes that lead to the development of cancer. It enables one to develop early diagnostic method to identify pre-cancer point or individuals with likely propensity to develop cancer. This method also provides an intermediate endpoint which can be used as an indicator of health and the likelihood of developing of cancer and the effect of a prophylactic therapy in addition to enabling one to develop animal models in which the effects of agents, genes, environmental factors, can be researched via their effects on the intermediate endpoint.

Given the traditional approach of diagnostic methodologies, it is truly surprising that specific histopathological markers could be determined and correlated a stage of cancer development with emphasis on the precancerous state. The new biomarkers are based on the observation that these focal changes are preceded by and/or coexist with subtle changes in the tissue housing the lesions.

Thus, the method of this invention comprises using biomarkers to predict the likelihood of cancer development according to the model presented herein, in the intestinal epithelium, which is the location of more than 90% of all cancers in the intestine. In this model, cancer, however scarce it may be in relation to total tissue mass, develops in relatively widespread precancerous populations present within the normal tissue side by side with normal cells. These populations arise from altered stem cells and possess some specific morphological and/or chemical features by which they can be recognized. Each of these specific population fits into and characterizes a stage of carcinogenesis.

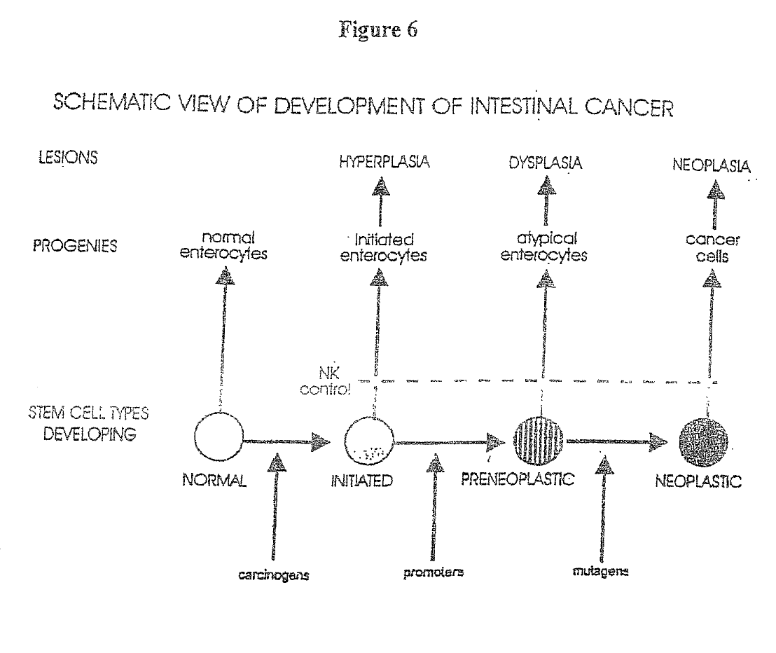

These stages are defined and are shown to arise in genetically predisposed individuals exposed to a particular group of environmental substances as shown in FIG. 6. Under the influence of carcinogens, the normal stem cells give rise to initiated stem (IS) cells. This high probability event takes place all along the intestinal tract. In some intestinal areas under the influence of promoters, some initiated stem cells transform into preneoplastic stem (PS) cells some of which in turn are prone to mutate and thereby to transform into neoplastic stem (NS) cells under the influence of mutagens. This last transformation is a low probability event. All these aberrant stem cells are recognized by the progenies they form and also by the lesions the progenies may form. The NK cells were found to affect the aberrant stem cells, not the normal ones. They can elicit the entrance of the aberrant stem cells into the Go stage so that these stem cells are inhibited from forming progenies or lesions even though they are present. This inhibitory influence is exerted by a factor produced by the NK cells. A further effect of the NK cells is the ability to kill the aberrant stem cells when activated by cytokines. The aberrant stem cells and their progeny are thus NK-dependent.

Lipid materials gradually accumulate in the enterocytes as they migrated along the crypts toward the colonic luminal surface. This accumulation occurs mainly in the apical cytoplasm of the enterocytes but could also take place in other cytoplasmic areas (e.g. infranuclear areas). In the course of the natural cell renewal process, these lipid material containing enterocytes reached the colon surface and eventually exfoliate into the colon lumen. They then break up and release their lipid material which then becomes part of the phospholipid content of the colonic mucus. Distinct glycoprotein containing mucus cells (goblet cells) also exfoliate and contribute to the glycoprotein portion of the colonic mucus. A third type of mucus secretory cell occupies most of the deep portions of the crypts: the “deep crypt secretory” (DCS) cells. These cells contain large numbers of prominent secretory granules which are secreted into the deep crypt lumen. These granules stain at least partially by the PAS (periodic acid Schiff) method, therefore they contain glycoprotein.

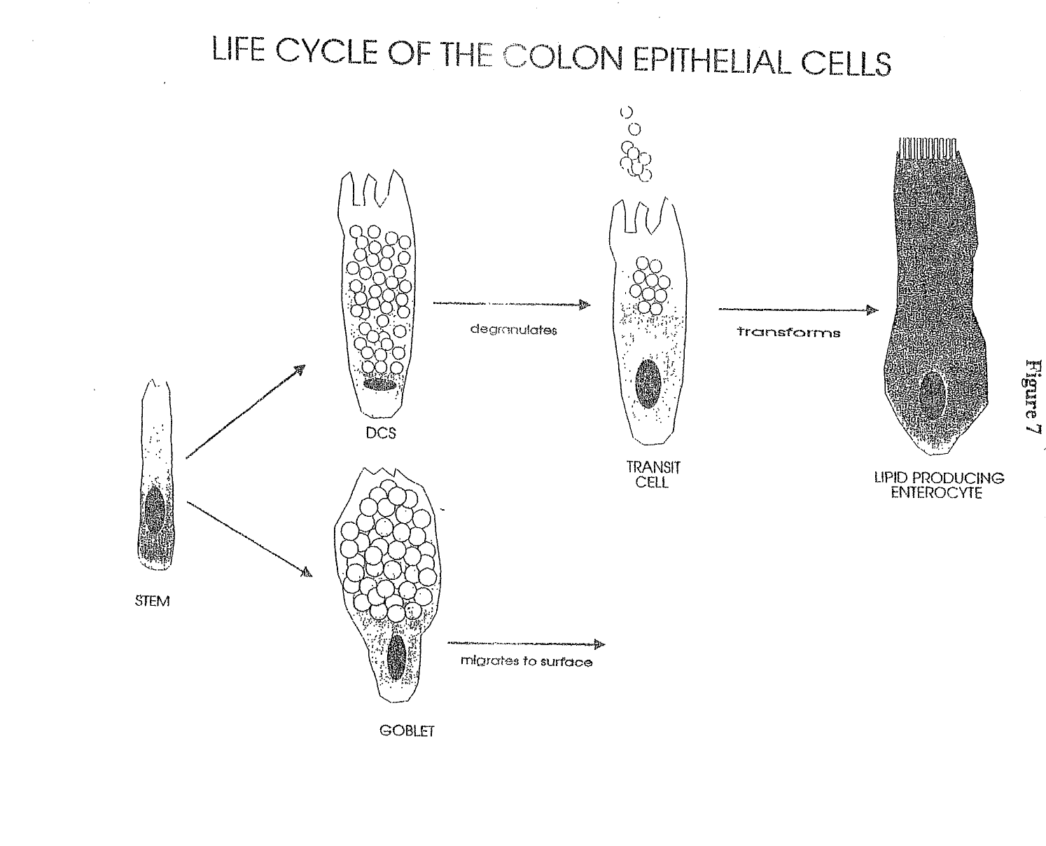

The interrelation between stem cells, enterocytes, DCS and goblet cells is illustrated in FIG. 7. The stem cells in the deep crypts first transform into cells containing numerous large secretory granules, that is DCS cells. On reaching about the midcrypt level, most granules are expelled (“degranulation”), the cells proliferate and then start to produce the phospholipids in the cytoplasm left free by the expelled granules. Electron microscopic evidence indicates that the endoplasmic reticulum is the site of the lipid material production, the lipid material being released and contained in small granules. Around the midcrypt level a metabolic switch takes place in enterocytes switching from glycoprotein to lipid material synthesis. This event is prominent morphologically.

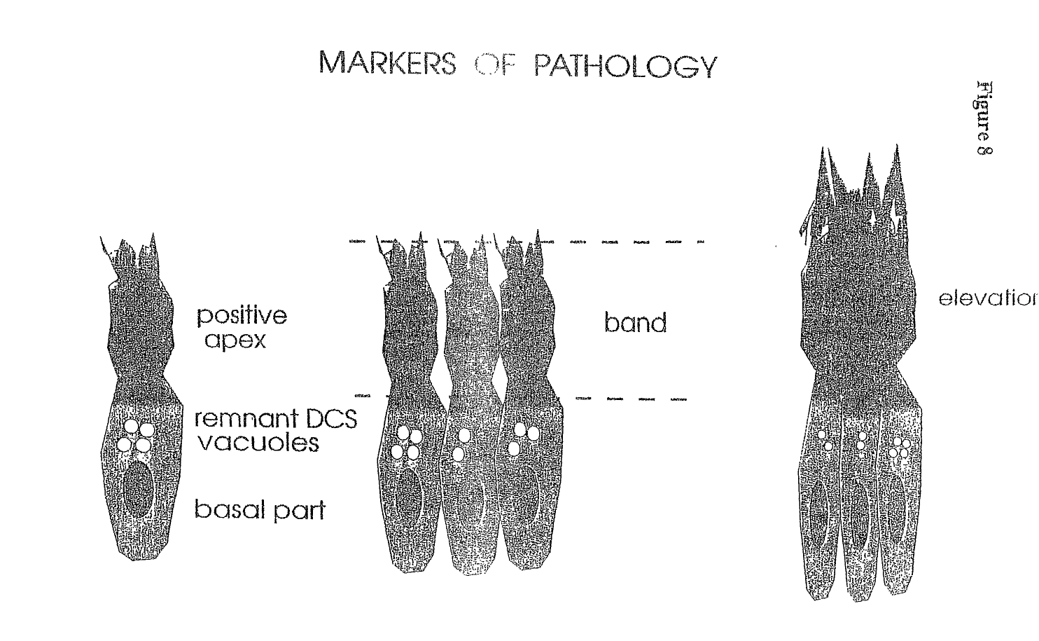

Some areas, especially around tumours, contain enterocytes with a high content of lipid materials, referred to as lipo-enterocytes or I-enterocytes (FIG. 8). Areas with a high probability of developing cancer show the lipo-enterocytes and associated pathological landmarks. These areas appear to be under the influence of a high concentration of promoters and therefore, the development of cancer is highly probable following exposure to promoters. The high lipoenterocyte content causes specific histopathological landmarks (FIG. 8) such as a surface band in epithelium positive for Schiff and showing special colour, prominent elevations on the surface of the epithelium. Further histological evidence showed that in highly promoted areas, stem cell number increases and the rate of formation of DCS cells is also elevated. These cells degranulate relatively early while still in the lower crypt. Consequently, large number of lipid material producing enterocytes are produced which spend more than average time in the crypts and therefore accumulate more than average amounts of lipid material. A high rate of DCS cell degranulation and the early metabolic switch from DCS cells to I-enterocytes are additional pathological markers of highly promoted areas.

The Method of the Invention

The present invention provides a means for assessing or identifying the stage of carcinogenesis for intestinal epithelial tissue. The sample used to use such method, may be a tissue sample such as biopsy regularly taken during colonoscopic examinations. There is a potential that the method can be developed for mucus samples, or for washings collected from the colon.

The first step of this method entails obtaining epithelial biopsies from several regions of the colon.

From the biopsies, histological preparations are made using standard procedures well known to one skilled in the art. Routinely the biopsies are embedded into paraffin or plastic blocks and then cut into about 5 micrometer thick sections. These sections are then mounted on glass slides, stained by immersing them into specific staining solutions and then examining them under the microscope.

The Activated I-Enterocyte Biomarker

This biomarker is promoted in high cancer risk areas and is recognized by the presence of active lipo-enterocytes and the associated pathological signs (elevations, early metabolic switch, apical band). This biomarker requires specific histological preparation of the samples so that lipids are preserved and then stained specifically. Frozen sections are prepared and treated by mild acid hydrolysis or by dilute solution of mercuric chloride. Aldehydes are liberated from the lipid materials in the lipo-enterocytes which are then reacted with Schiff reagent. The mercuric pretreatment has a further advantage in that it leaves mercuric ions built into the lipid material. The lipid material can then be visualized thereafter by any of the conventional mercury stains. The lipo-enterocytes stained by the Schiff reagent have provided special pathological markers which in turn allow for the recognition of high risk sites as described earlier. The Schiff positive atypical band is most conspicuous and the elevations are most frequent in highly positive areas of the tissue. In such positive sites, the changeover from DCS cells to lipo-enterocytes takes place lower in the crypt than normal, ie. earlier in the process of cell renewal. The site of the changeover is thus lower in the crypts that normal. These crypts also have a higher density of lipo-enterocytes towards the surface so that goblet cells tend to exfoliate in these areas while still in the upper crypt. These markers can therefore be used in the recognition of “promoted” or “high risk” sites.

This biomarker can also be expressed in a quantitative manner as the percent area occupied by Schiff positive lipid material within the boundaries of the epithelium in representative histological sections. There is a normal level of expression which is around 15-20%, and there is an abnormal level of expression in promoted areas, approaching twice the normal level of expression. These percent values can be determined by computerized analysis of microscopic images. In highly positive areas, there is also a higher density of staining and there is some shift in the colour. Higher percent values indicate stronger promoting influence and therefore a higher risk of the patient developing cancer. The observations demonstrate that enterocytes under stimulation by promoters or promoter-like substances produce higher than normal lipid material in proportion to the intensity of the stimulus. The amount and intensity of the Schiff positive content of the enterocytes of the surface epithelium thus reflects the intensity of the stimulus. In the microscopic images using representative segments of the surface epithelium are outlined. Also, the area of the Schiff positive regions within the epithelium is outlined. The ratio of the two types of areas is then expressed as a percentage, or in any other arbitrary units. This can be done by any method of image analysis, including computerized ones. An index of positivity can thereby be constructed. Measurements of staining intensity can also be included in such an index. The staining of the intestinal tissue by the Schiff reagent imparts a pink colour to the tissue, including the epithelium. The truly Schiff positive areas stain magenta and deep magenta in the high density areas. Such colour differences can be accented by computerized methods.

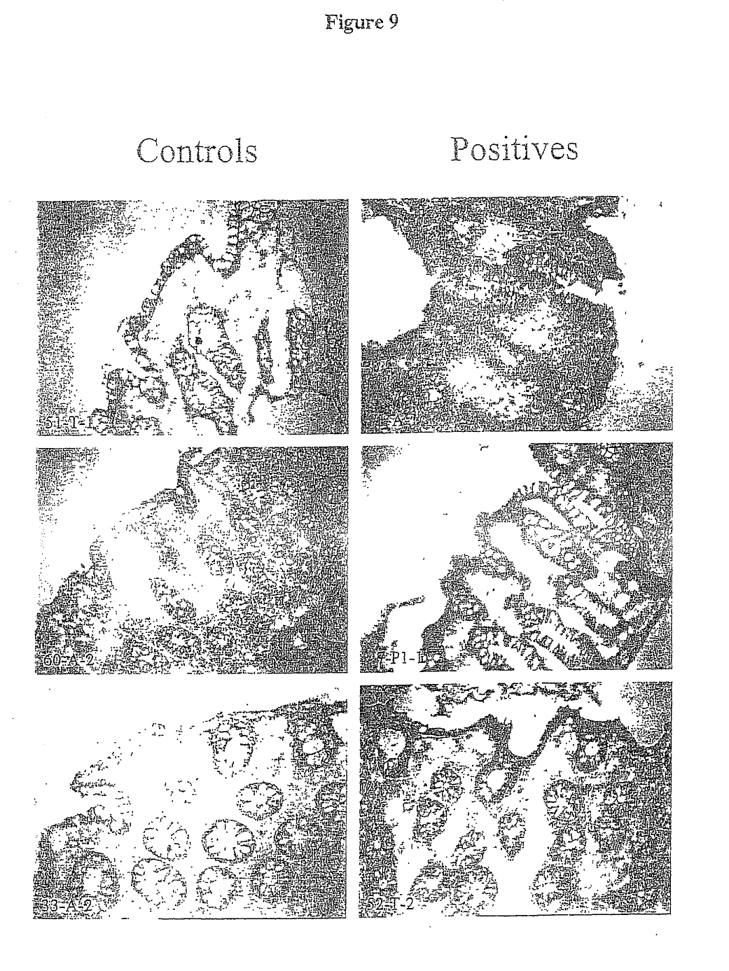

The activated I-enterocyte is a histopathological marker requiring that tissue samples or biopsies be taken mainly from the epithelial lining of the intestine. These samples are to undergo histological processing and the histological preparations then are subjected to microscopic analysis. This analysis may be carried out through the microscope or on the photographic or computerized image of the sample made under the microscope. Routine analysis of biopsies with the staining for lipid materials can mark out the high cancer-probability areas in the colon as shown in FIG. 9. Image analysis can also assign a numerical value to these samples expressing the relative proportion and intensity of lipid material containing areas in the epithelium.

The Method Embodied in a Kit

Kits to work the method are also embodiments of this invention. The materials for use in the method of the invention are ideally suited for the preparation of a kit. Such a kit may comprise a carrier means compartmentalized to receive in close confinement one or more container means, such as vials, tubes, and the like, each of the container means comprising one of the separate elements to be used in the method. Components of the kits would include specific materials necessary to work the methods. Reagents for fixing the tissue samples would be included in the kits. For example, the kits could contain, within separate container means, fixatives such as a dilute acid or mercuric chloride fixatives, that enable chemical preparation of the histological sections. In an alternative procedure, the biopsies can be frozen, so kits using this procedure would not contain chemical fixatives.

The frozen or chemically fixed histological sections are placed on specially coated slides to preserve their integrity during processing, so specially coated slides that would sufficiently adhere the samples to the glass slide would be provided. Plastic or polymer slides may also be used.

Various pre-measured reagents would be provided within separate container means. One example of such reagents is a specialized Schiff reagent, which would be standardized so that optimal and standard colours yield upon development. Another reagent would be the silver intensifier solutions and/or physical and chemical developer solutions would be provided, which surround the mercury built into the tissue (at lipid material sites) with silver atoms. This is an alternative way to visualize the lipid material. Alternatively, other solutions may be provided which provide mercury-staining. Newly developed stains might also be included in these kits.

Finally, material items such as slides, cover slips and mounting media would be provided to convert the stained slides to permanent preparations.

Advantages of the Invention

The method of this invention is particularly important for determining the promotion stage of cancer development, as demonstrated in FIG. 9. Current techniques can only determine the presence of cancer and determine its stage of development to some extent. In contrast, the method of this invention provides a means to determine the propensity of the tissue to develop cancer in addition to providing information relative to the particular etiology in that patient.

This method of using biomarkers to assess the biological status of cancer development: 1) enables one to study the effects of different therapeutic regimes to design the most effective course of chemotherapeutic or radio-therapeutic treatment for a patient; 2) provides a means for studying the underlying cause/changes that lead to the development of cancer; 3) enables one to develop early diagnostic method to identify pre-cancer point or individuals with likely propensity to develop cancer; 4) provides an intermediate endpoint which can be used as an indicator of health and the likelihood of developing of cancer and the effect of a prophylactic therapy; 5) enables one to develop animal models in which the effects of agents, genes, environmental factors, can be researched via their effects on the intermediate endpoint.

Moreover, the method of this invention allows the practitioner to distinguish between cellular changes associated with inflammatory diseases which are not life-threatening and carcinomatous changes which are life-threatening or may progress to be life-threatening.

To assist in understanding the current invention, the following non-limiting examples are provided.

The following examples should not be construed as specifically limiting the present invention, variations presently known or later developed, which would be in the understanding of one skilled in the art and considered to fall within the scope of the present invention as described herein.

### EXAMPLES

### EXAMPLE I

### Preparation of Fresh Colonic Biopsies for Light Microscopic Examination

Biopsies are frozen on dry ice and sectioned by cryotome in proper longitudinal orientation. The sections are mounted on glass slides and stored in the freezer.

To stain, the slides are taken from the freezer and thawed briefly. The sections are treated with a proper mordanting solution that oxidise specific lipids so that some aldehyde compounds are released to allow for staining with Schiff reagent. The sections are mordanted with 1% mercuric chloride or with 6M hydrochloric acid for about one minute, followed by rinsing. The sections are stained with Schiff reagent for approximately five minutes. The colour is developed in running water for 15 minutes. The samples are then rinsed, dried and a cover slip is applied for examination.

### EXAMPLE II

### Demonstrating the use of the Activated L-enterocyte Biomarker

The method using biomarker 3 was employed in a study involving 80 patients, wherein an average of 4 frozen samples were collected from each case. The frozen sections were stained using the method of the invention and analysed. Among the 80 patients who had former colon cancer, who were having colon cancer and those with frequent occurrence of colon cancer in the family, all showed biopsies which were predominantly positive for biomarker 3. This study demonstrates that a positive reading indicates “the proneness” to develop cancer.

Biopsies were taken from the colonic mucosa at various regions of the colon. The biopsy is a piece of the mucosa of about 1-2 mm diameter. The biopsy is dropped into prepared fixative such as formalin.

In this example, the biopsy was placed flattened into O.C.T. compound in a small aluminum dish and was then quickly frozen on dry ice. More O.C.T. compound was then added to enlarge the frozen block. Using a freezing microtome, 8-10 micrometer thick sections were then cut and placed on a glass slide and the whole preparation was then kept frozen.

In preparation for staining, the slides were quickly thawed and one drop of 1% mercuric chloride solution was placed over the sections for 1 minute. The slides were quickly rinsed and then stained in Schiff solution for 10 minutes. The slides were then placed in running tap water for 10 minutes and then dried and cover slipped.

The resulting slides were examined under the microscope and the relevant images were captured in an image analysis software for later analysis. Using the markers listed earlier, the samples were assessed either as positive or negative and compared to the diagnosis made by other means by the gasteroenterologist.

Positive tissue was always associated with individuals whose colon contained cancer at the time of biopsy or had cancer earlier, or the family had frequent occurrence of colon cancer. In the positive colons, there was thus some agent which made the probability of cancer high. Diverticulitis and ulcerative colitis were also positive, but there were other markers which could distinguish these cases.

The results indicated that in general, highly cancer prone cases demonstrated a strong staining, most prominent in the colonic surface epithelium. Here, in addition to the general light background staining, a strong staining component was revealed which could be enhanced to some degree by computerized imaging methods. In the most serious cases, most surface epithelium had the magenta component and this component was accumulating so much that there were bumps on the surface. This accumulation of the magenta component is the critical histopathological marker.

The degree of staining is proportional to the degree of propensity to develop cancer so the results can be collected and analysed using a computerized method of evaluating the relative amount of the magenta area. Thus, a computer measured reading can be compared against a background reading to indicate an quantitated value for the propensity of carcinogenesis.

Visual analysis of the representative images obtained from the 80 patient study indicate the following distinct patterns:

1. No positive reading: slices showing mucosa not involved in any of the traditional lesions;

2. Low positive reading: slices are results from colons which have a condition not related to cancer, as for example, stricture, diverticulitis, etc. These samples reveal a low level of positive material in the surface epithelium. Such low levels may also be found in cancer or polyp bearing colons in some but not in the majority of samples. This in turn indicates that the promoter responsible is not of uniform distribution;

3. Variable positive reading: polyp bearing colons display variably sized but sometimes heavy positive areas. It is indicated that polyps arise in such heavily promoted areas. Slices taken from regions of former cancer: a good number of areas show low positivity. Some heavy positivity however, is still shown at many places indicating that although the tumour was removed, the promoting environment was not cleared up;

4. Heavy positive reading: slices wherein a large proportion of the surface epithelial areas shows heavy positivity. Crypts at many places also show positivity indicating that response to promoters start early in the life of the enterocytes in these cases.

FIG. 9 is exemplary of the findings. Positive samples have clearly shown a clear and prominent apical band on the surface of the colon, i.e. in the surface epithelium. In colour photographs, there is a deep magenta layer. In negative samples, this layer is thinner, and pinkish, very much as the rest of the tissue stained nonspecifically by Schiff. Positive areas stain with the specific magenta colour.

### EXAMPLE III

### Quantitative Analysis of the Activated Enterocytes

Biopsies were obtained from 24 cases, including 3 cases of ulcerative colitis and one of diverticulitis. Four are still to be studied. Among the remaining 16 cases, 11 positive readings and 5 negative readings were determined using the histological method of visualizing activated enterocytes. The microscopic images were converted into digital images which were then analysed for the percentage of activated enterocytes in the epithelium and in the crypts. The staining was performed by first oxidizing the specimens and then staining with Schiff reagent on frozen specimens. The images were stored in computer and were evaluated using the Northern Exposure software. The boundaries of the epithelium were traced. In this area, some of the cells were carrying a heavy load of the staining substance (plasmalogen). The computer was adjusted so that it measured the percentage of the areas within the surface epithelium.

### EXAMPLE IV

### Demonstration of Method

I. Collecting Histological Samples

Fresh colon tissue was obtained from surgical resections and from biopsies obtained during endoscopic examinations. The tissue was embedded into O.C.T. compound and then frozen on dry ice. Frozen sections were cut at 9-10 micrometer thickness, attached to glass slides and then kept frozen in a humid atmosphere.

The samples collected represented various disease categories:

I tumor-bearing colon

II polyp-bearing colon

III former cancer in the colon

IV former polyp in the colon

V family history of cancer or polyp of colon

VI various non-cancerous cases (obstruction, nonspecific inflammation, etc.)

We took several samples from each case (Kiernan, J. A., Histological and Histochemical Methods: Theory and Practice, 2^{nd} edn. Oxford: Pergamon Press, 1990; Rapport, M. M., Lipid Res. 1984; 25: 1522-1527; Altmann, G. G., Amer. J. Anat. 1983; 167: 95-117; Altmann, G. G., Electron Microsc. Tech. 1990; 16: 2-14; Chang, W. W. L., et al., Am J. Anat. 1971; 131: 73-99; Altmann, G. G., Epith. Cell. Biol. 1995; 4:171-183; Terner, J. Y., et al., Stain Technol. 1961; 36: 265-278) depending on availability. Only occasional samples were taken from the tumours or the polyps themselves. We were mostly interested in the non-involved so called “normal” mucosa. It was taken at various distances along the available colon pieces and when possible, at various given distances from the tumour.

II. Processing of the Histological Samples

The sections were thawed by slight finger pressure against the glass side. The sections were then flooded with a 1% solution of mercuric chloride for 1 minute. The sections were then briefly washed in distilled water then flooded with Schiff solution for 5 minutes. The Schiff reagent was then washed out, the sections were washed in running tap water for further 5-10 minutes, then dryed and coverslipped.

III. Qualitive Evaluation of the Samples

The histochemical reaction we used is specific for plasmalogens (Kiernan, J. A., Histological and Histochemical Methods: Theory and Practice, 2^{nd} edn. Oxford: Pergamon Press, 1990; Terner, J. Y., et al., Stain Technol. 1961; 36: 265-278). It is based on the conversion of the ether bond by attachment of mercury and subsequent formation of aldehyde which is then stained by the Schiff reagent. The histochemical procedure had to preserve lipids and was only effective when the tissue was pretreated with the mercury solution. The Schiff reagent provided for an overall pinkish staining of the tissue so that there was no need for counterstaining. The actual positive substance was magenta, that is having at least one color component additional to pink.

The Schiff positive substance was confined to the cytoplasm in enterocytes and was gradually spreading as the enterocytes matured in the course of their natural life cycle. The extent of accumulation of plasmalogen was assessed in the enterocytes, those that were heavily laden with plasmalogen were termed “lipo” or “1” enterocytes.

IV. Histometry

The spread of the plasmalogen within the enterocytes was measured in the surface epithelium. in the histological images. Representative epithelial areas were selected. Total such area versus area occupied by plasmalogen was then quantified by area measurements. The percent of enterocyte images occupied by plasmalogen was called the plasmalogen index (PI).

V. Cytological Assessments

The colon epithelium is continually renewed by new cells arising from the stem cells of the crypt bases. The new cells go through various stages and give rise to various derivatives (e.g. 6). In order to evaluate these lineages, more cytological detail than provided by frozen sections was needed. For these purpose, several samples were taken in addition to the frozen ones. Also, some colon samples were obtained from mice and pigs. The samples were fixed by Carnoy's fluid and/or by a mixture of osmium tetroxide and potassium permanganate (Kiernan, J. A., Histological and Histochemical Methods: Theory and Practice, 2^{nd} edn. Oxford: Pergamon Press, 1990). After fixation for 2-4 hours, the fixative was washed out and the samples were embedded in plastic (Historesin). Sections were cut at 1-2 micrometers and stained with toluidine blue or with iron hematoxylin (Kiernan, J. A., Histological and Histochemical Methods: Theory and Practice, 2^{nd} edn. Oxford: Pergamon Press, 1990).

Results

Plasmalogen is the Source of Schiff Positivity

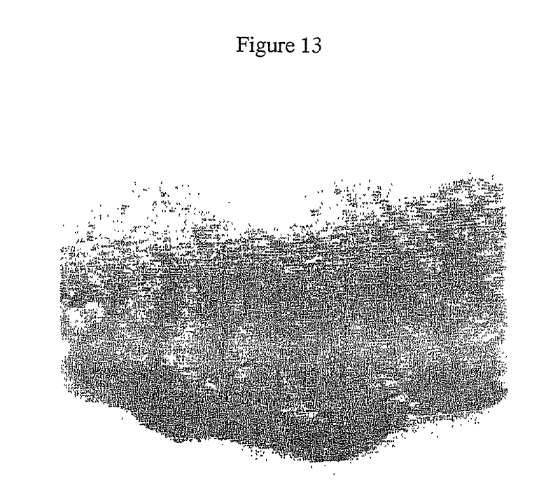

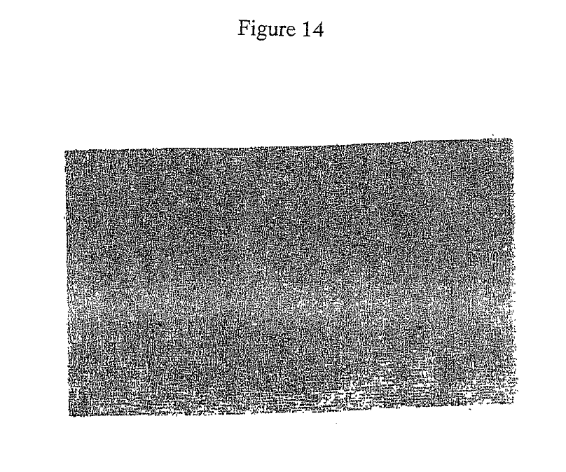

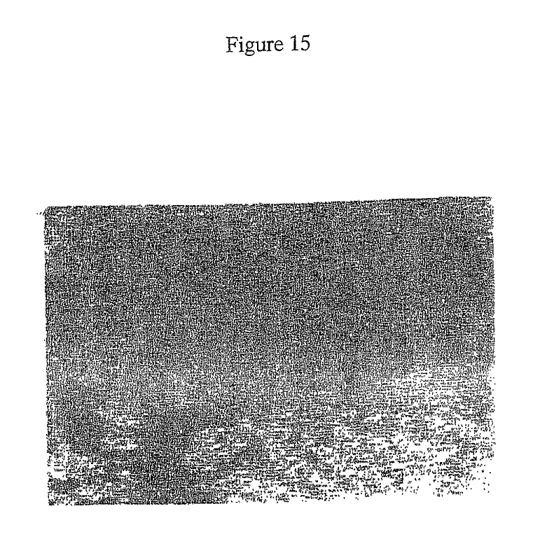

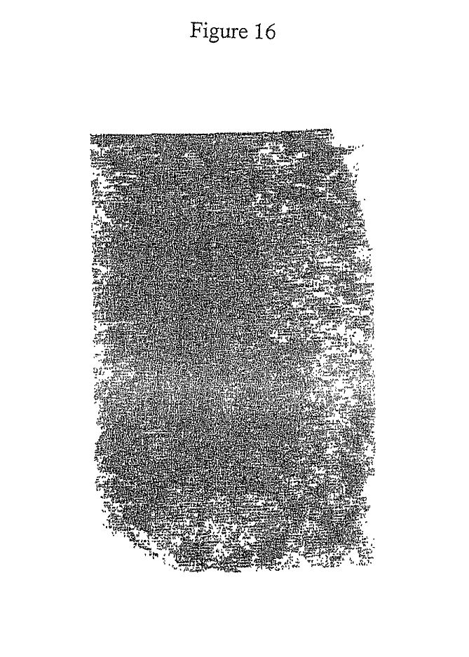

Upon sixty years of experience of histochemists with the Schiff reagent, it has been found to be a reliable indicator of the presence of aldehydes (Kiernan, J. A., Histological and Histochemical Methods: Theory and Practice, 2^{nd} edn. Oxford: Pergamon Press, 1990). In general, oxidation of carbohydrates by periodic acid and oxidation of plasmalogens by mercuric chloride lead to aldehydes demonstrable by conversion of the colourless Schiff reagent into magenta colour. Clear cut histochemical demonstration techniques have thereby been provided for these two types of substances (Kiernan, J. A., Histological and Histochemical Methods: Theory and Practice, 2^{nd} edn. Oxford: Pergamon Press, 1990). In view of Yeung's finding of Schiff positive mucus in human rectum, we tried to identify Schiff positive structures in frozen samples of human colonic mucosa. In general, no such structures were found, the frozen sections remained unreactive in the presence of Schiff. With prior periodic acid oxidation, for example, the mucus content of the goblet and the DCS cells stained. The DCS (“deep crypt secretory”) cells were first demonstrated in our laboratory (Altmann, G. G., Amer. J. Anat. 1983; 167: 95-117; Altmann, G. G., Electron Microsc. Tech. 1990; 16: 2-14) as cells somewhat similar to goblet cells but accumulating in the deep crypts where they tend eventually to release their glycoprotein granules en mass leading to a phenomenon which we referred to as “deep crypt secretion”. To our surprise, the mercuric chloride pretreatment brought out entirely different structures without demonstrating goblet or DCS cells. Several of the epithelial absorptive cells or “enterocytes” showed highly Schiff positive areas (FIG. 13-16). According to the histochemical evidence, these areas consisted of plasmalogens, the colonic occurrence and location of these substances being unknown at the time. These substances were furthermore soluble in lipid solvents as frozen sections were needed to preserve them. Pilot EM evidence indicated that they were produced in the cytoplasmic endoplasmic reticulum and were therefore of phospholipid nature.

Relation to the Renewal of the Enterocytes

Following the well elaborated concept of the renewal of enterocytes (Chang, W. W. L., et al., Am J. Anat. 1971; 131: 73-99), no enterocyte is of more than 6 days old in the colonic epithelium. The average life span of these cells is about 6 days after which they exfoliate from the epithelium and are being replaced by younger cells forming from the mitosis and the phenotypic transformation of stem cells and derivatives. Positional analysis of cells along the crypt axis is a reliable source of the age of the enterocytes as with age they migrate and occupy higher positions. Plasmalogen patches appear as a rule in young enterocytes at the lower crypt level. At this time, the patch is mostly infranuclear but some parts may already be partially supranuclear. With age, the patches appear to expand and fill up more and more of the upper cytoplasm of the cells. When this apical content reaches quite an extent, the cell apex may appear as a lipid containing elevation in the otherwise normal looking epithelium (FIG. 8). Our general conclusion was that practically all enterocytes entered a plasmalogen-producing phase towards the end of their life cycle. However, the amount of plasmalogen produced varied in a patchy fashion that is in some colonic areas, the cells were relatively low producing whereas in some regions they were very high producing. The possible reasons will be elaborated below.

The Histometric Measurements

These measurements have confirmed the observation that some enterocytes are low plasmalogen producers whereas in others, this production is high (Table 1). The histological observations have indicated furthermore that when the enterocytes exfoliate at the end of their life span, the plasmalogen content is carried with them for a short while but soon the cells break up and release the content the colonic mucus. The colonic mucus thus appears to be composed of goblet and DCS cell secretions which are most probably glycoproteins and also of phospholipids, the latter coming mainly from enterocytes in the above described manner.

The results on the individual samples are shown in Table 1. from 10 to 30% plasmalogen index may be considered as the normal range. An index between 30 and 40% may be considered as moderately elevated. Above 40%, it is considered as high.

Considering the polyp-bearing colons, the average plasmalogen index was the highest, 39.6%. More than half of the samples (54%) had high index. About 17% of the samples had moderately elevated index.

Considering the cancer-bearing colons, the average index was much lower, 22.6%. Only 9% of the samples were in the high category, and 17% in the elevated one.

Considering the colons with former cancer, the average index was slightly elevated, 30.5%. About 15% of the samples were in the high category and 26% were in the moderately elevated category. In the colons in families with history of cancer or polyp, the average index was 25.9%. About 11% of the samples were in the high index category, and 26% in the moderately elevated category.

In the non-cancerous group, most values were in the normal range, the average index being at 22.7%. About 10% of them were moderately elevated with practically none in the high range.

In conclusion, high plasmalogen indices with high occurrence were exclusively in cancer-bearing colons. Poly-bearing colon had a few but much less of these high indices. In the case of former cancer or polyp, several samples were moderately elevated but most were within normal range.

In conclusion, the results have shown that the tendency of having highly elevated plasmalogen content was very high in the cancer-bearing colons. This tendency was much more moderate in the other groups but some elevation still lingered after removal of tumours or polyps or even when the tumours were only in the family.

Considering the position of various samples along the colon, the distribution of the positive sample was patchy, only some were seen in close proximity of tumours, others were at quite a distance. In other words, positivity did not mean that there was an overall stimulatory factor present which stimulated all enterocytes. The positive enterocytes did occur in groups so that they were predominant in a given sample but positive and negative samples alternated.

Cytological Events

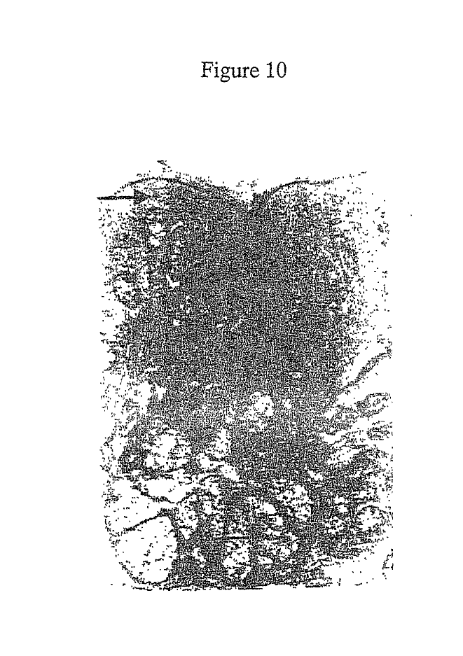

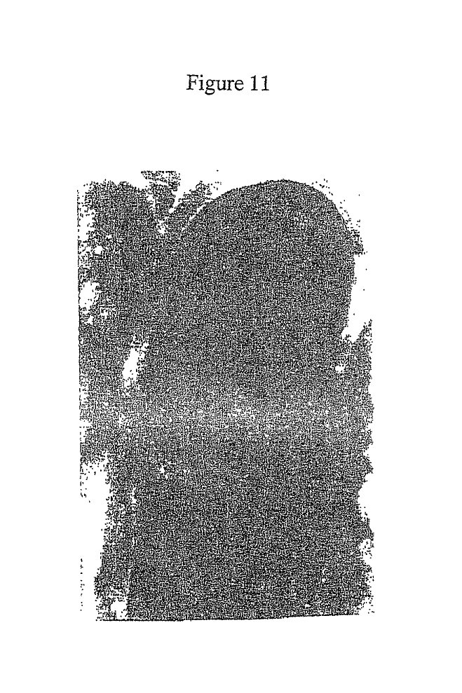

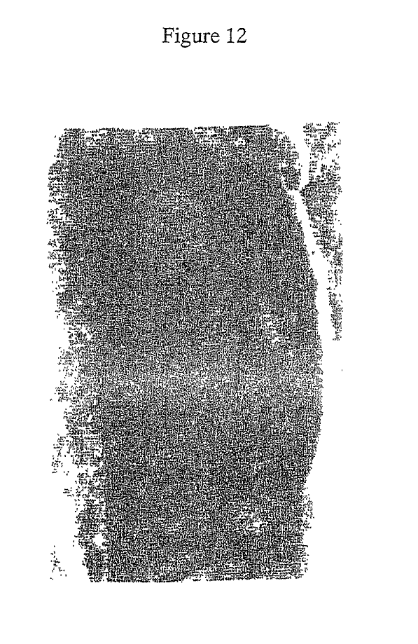

The plasmalogen production by enterocytes and the connection of this phenomenon to cancer development are new findings. The semithin histological material along with already published results (Altmann, G. G., Amer. J. Anat. 1983; 167: 95-117; Altmann, G. G., Electron Microsc. Tech. 1990; 16: 2-14) are sufficient to decipher the main cytological stages in reaching the plasmalogen producing stage (FIGS. 8, 10-12, and 16). As shown by the semithin sectioned material (from human, pig and mouse), the immediate stem cell derivatives become filled with prominent secretory granules. These are the deep crypt secretory (DCS ) cells which we have reported earlier (Altmann, G. G., Amer. J. Anat. 1983; 167: 95-117; Altmann, G. G., Electron Microsc. Tech. 1990; 16: 2-14). These cells migrate along the crypt axis and lose their granules into the crypt lumen via a process which we call “deep crypt secretion”. After most of these granules are lost, the cells enter a brief period of mitosis and then the transit stage when absorptive and plasmalogen-producing properties develop. On light microscopic examination, these enterocytes are large cells much of the apical cytoplasm is being filled with plasmalogen (FIGS. 8 and 13-16). This is the type of cell which is finally extrudes and contributes its plasmalogen to the colonic mucus.

In short, the DCS cells are the immediate precursors of the plasmalogen producing enterocytes, the loss of their secretory granules (“deep crypt secretion”) is being the critical and well visible event. It is to be acknowledged at this point that the presence of a vacuolated cell type was notice earlier in the rodent ileum (Chang, W. W. L., et al., Am J. Anat. 1971; 131: 73-99), the loss of the vacuoles leading to the transit enterocytes. It seems that in other regions of the colon, especially in the ascending region, this phenomenon is much more prevalent involving the much more prominent DCS cells and the phenomenon of deep crypt secretion which may be quite impressive (Altmann, G. G., Amer. J. Anat. 1983; 167: 95-117).

Discussion

Plasmalogens, a class of phospholipids with an unsaturated ether bond, are still enigmatic as to their exact biological role. They are known to occur in a number of tissues (e.g. nerve, muscle) (Rapport, M. M., Lipid Res. 1984; 25: 1522-1527). This is the first report on their occurrence in the colon epithelium and that they are produced by the enterocytes. The latter cells are also called absorptive epithelial cells indicating that their main role was thought to be in absorbing water from the faecal content. As our observations indicate, an additional and rather significant role would be the production and accumulation of plasmalogens and after their exfoliation, adding these accumulated phospholipids to the colonic mucus. The single main source of colonic mucus has been thought to be the glycoprotein secretion from the goblet cells. We confirm here our previous report (Altmann, G. G., Amer. J. Anat. 1983; 167: 95-117; Altmann, G. G., Electron Microsc. Tech. 1990; 16: 2-14) that DCS cell secretions are also added to the mucus and we report for the first time that plasmalogen-type phospholipids are also added from the enterocytes. It was surprising to see how large the enterocytes were when their phospholipid content was preserved in the histological preparations. This was suggestive that the contribution of these cells to phospholipid metabolism may indeed be quite substantial. The histochemical basis of demonstrating plasmalogens has been worked out (Terner, J. Y., et al., Stain Technol. 1961; 36: 265-278). Mercury is built into the double bond of the ether linkage. There is an intermediate product which soon dissociates into an alcohol and an aldehyde. The aldehyde is then demonstrated by its reaction with the Schiff reagent. By histochemical definition then, the areas seen to be stained magenta were plasmalogens. There was unspecific pink tissue staining as well which provided an overall differentiation of tissue parts without the need for counterstaining.

Traditionally, the epithelium of the colon is regarded as one of the fastest renewing cell populations of the body: stem cells produce transit cells that embark on 3 possible lines of differentiation, enterocyte-line, goblet cell line, and enteroendocrine cell line (Chang, W. W. L., et al., Am J. Anat. 1971; 131: 73-99). Our histological evidence indicates that the enterocyte-line is more complex as intermediary cell types are interposed. Such a cell type, called “vaculoated cell” was recognized a while ago (Chang, W. W. L., et al., Am J. Anat. 1971; 131: 73-99) and was postulated that the immediate stem cell derivative acquired vacuoles which gradually disappeared before mitosis and differentiation into enterocyte took place. This phenomenon is indeed observable in many areas of the colon. As we have observed however, in many colonic areas and under various circumstances, this phenomenon is much more intensive, the immediate stem cell derivative fills up with prominent secretory granules which eventually exocytose, this often happening en mass. We refer to the secretory cells so formed as DCS cells and to the exocytosis of their granules as deep crypt secretion. This secretion apparently fills the lower crypt lumen with a special type of glycoprotein different in its staining properties from that of the goblet cells. The cells mitose and then differentiate soon after this event. A pilot study of ours with the electron microscope (to be published separately) has shown that as the secretory granules disappear from the cytoplasm, osmiophilic areas of endoplasmic reticulum appear which apparently produce the plasmalogens well demonstrated in parallel light microscopic preparations. The various observations indicated furthermore that there are two levels of plasmalogen content in enterocytes: normal and elevated. The elevated seems to be in groups of enterocytes that are prevalent in “cancer prone” areas of the colon. Cancer proneness has a specific definition derived from another extensive line of investigation in our laboratory (summarized in Altmann, G. G., Epith. Cell. Biol. 1995; 4:171-183). In this work on experimental carcinogenesis, we have built up a working model of intestinal cancers at will and within weeks and progression of precancer to cancer could also be halted. The work has shown that practically all individuals predisposed genetically to intestinal cancer harbour a small percentage (about 10%) of slightly changed “initiated” epithelial cells. The presence of these cells in itself is harmless but these cells if exposed long enough to another group of substances, “promoters”, they become sensitized to mutagens so that in the presence of mutagens they are likely to transform into cancer cells. Even one such cell can establish the disease. We found, furthermore, that long enough prior exposure to promoters was needed without which such transformation into cancer cells did not take place. Promotion was thus found to be a key event in carcinogenesis. Promoters in the intestine are known to derive from bile and/or the bacterial flora. They tend to accumulate in some parts of the intestine making these parts prone to cancer formation. Statistical and experimental evidence is accumulating that these areas indeed are the ones that respond with the elevated plasmalogen production. The enterocytes involved are probably the initiated cells which tend to proliferate locally in the presence of promoters.

It may be concluded that the plasmalogen reaction described in the present work, if made on intestinal biopsies, provides a new histopathological tool by which the presence and the location of cancer prone areas can be determined. Ways devised to clear these areas from promoters may also lead to new methods of reducing cancer risk. Since the enterocytes accumulate plasmalogen mainly in their apical portion, a plasmalogen-film is present in those areas of the colonic surface that are lined by enterocytes (FIG. 8). Some areas are lined by groups of goblet cells but these groups are in minority especially in high risk areas. Instead of using just histopatholgical tests, there can be ways of measuring the thickness of the plasmalogen-film itself thereby outlining the location of the high risk areas. In all, new aspects of enterocyte metabolism have surfaced in the present work and they seem to be closely connected with some aspects of carcinogenesis. This may eventually lead us to clues to the connection between normal and carcinogenic metabolism.

A further implication of the connection with promoters is that the protein kinase C system may be involved. Connection of promoters with the protein kinase C regulation of mitosis as well as with phospholipid turnover has been reported (DeRubertis, F. R., et al., C. Prev. Med. 1987; 16: 572-579; Lafave, L. M. Z., et al., Lipids 1994; 29: 693-700). It is thus probable that this newly found “plasmalogen phenomenon” together with other findings on carcinogenesis and phospholipid metabolism may provide new insights into mitotic regulation and the cancer problem itself.

The embodiments of the invention in which an exclusive property or privilege is claimed are defined as follows:

1. A method of determining whether or not an animal or human is at risk of developing a pathology comprising the steps of:
(i) obtaining a sample from an animal or from a human source;
(ii) determining if said sample comprises activated I-enterocytes; and
(iii) concluding if the animal or human is at risk of developing colorectal cancer.

2. A method according to claim 1 for determining if the animal or human is at risk of developing a neoplasia or a cancer pathology.

3. A method according to claim 2 for determining if the animal or human is at risk of developing a colorectal cancer pathology.

4. A method according to anyone of claims 1 to 3, characterised in that the sample obtained in step (i), is an epithelial tissue.

5. A method of determining whether or not an individual is at risk of developing a colorectal cancer comprising the steps of:
(i) obtaining a sample from the individual;
(ii) determining if said sample comprises activated I-enterocytes; and
(iii) concluding if the individual is at risk of developing colorectal cancer.

6. A method of determining whether or not an individual is at risk of developing a colorectal cancer, according to claim 5, characterised in that the sample is an epithelial sample.

7. A method of determining whether or not an individual is at risk of developing a colorectal cancer according to claim 6, characterised in that the epithelial sample is selected in the group constituted by intestinal tissues, mucus samples and sample of washings collected from the colon.

8. A method of determining whether or not an individual is at risk of developing colorectal cancer according to claim 6 or 7, wherein in step (i) the sample of epithelial tissue is recovered by biopsy, by surgical excision or by mucosal scrapping.

9. A method of determining whether or not an individual is at risk of developing colorectal cancer according to anyone of claims 1 to 8, wherein in step (ii) the activated I-enterocytes is selected in the group constituted by I-enterocytes with altered morphology.

10. A method of determining whether or not an individual is at risk of developing colorectal cancer according to claim 9, wherein the I-enterocytes with altered morphology are I-enterocytes with aprical abnormalities.

11. A method of determining whether or not an individual is at risk of developing colorectal cancer according to claim 10, wherein the I-enterocytes with aprical abnormalities are I-enterocytes with exaggerated phospholipid formation in areas where cancer promoters are accumulating.

12. A method of determining whether or not an individual is at risk of developing colorectal cancer according to anyone of claims 7 to 11, wherein in step (ii) the intestinal tissue recovered by biopsy is embedded into paraffin or plastic blocks and then cut into thick sections.

13. A method of determining whether or not an individual is at risk of developing colorectal cancer according to claim 12, wherein in step (ii) the intestinal tissue recovered by biopsy is embedded into paraffin or plastic blocks and then cut into about 5 micrometer thick sections.

14. A method of determining whether or not an individual is at risk of developing colorectal cancer according to claim 12 or 13, wherein in step (ii) said thick sections are mounted on glass slides, stained by immersing them into specific staining solutions and then examined under the microscope for the presence of activated I-enterocytes.

15. A method of determining whether or not an individual is at risk of developing colorectal cancer according to claim 12 or 13, wherein the thick sections are prepared and treated by mild acid hydrolysis or by dilute solution of mercuric chloride before characterisation with a Schiff's reagent in order to obtain a activated I-enterocytes biomarker.

16. A method of determining whether or not an individual is at risk of developing a pathology according to anyone of claims 1 to 15, wherein it is concluded in step (iii) that:
the animal or human is classified at risk of developing cancer if a deep magenta colouring may be detected in step (ii) in any part of the sample; and
the animal or human is classified at no risk of developing a pathology if no deep magenta may be detected in the sample prepared in step (ii).

17. A method of determining the stage of pathology in an animal or human comprising the steps of:
(i) obtaining a sample from the animal or from the human, which sample is preferably selected in the group constituted by samples of intestinal tissue, mucus samples and samples of washings collected from the colon;
(ii) determining if said sample comprises elevated levels of activated I-enterocytes; and
(iii) quantifying the elevated level of activated I-enterocytes and correlating said elevated level of activated 1-enterocytes to the stage of pathology.

18. A method according to claim 17, wherein the pathology is a neoplasia or a cancer pathology such as a colorectal cancer.

19. A method of determining the stage of cancer in a patient with colorectal cancer according to claim 18, wherein a sample of intestinal tissue is, after staining by a Schiffs reagent or after staining by a modified Schiff's reagent, evaluated for its level of activated 1-enterocytes which is proportional to the percentage area of the sample showing a deep magenta colouring.

20. A method of determining the stage of cancer in a patient with colorectal cancer according to claim 18, characterised in that the level of activated I-enterocytes is qualify of high if more than 30% of the area of the sample shows a deep magenta colouring.

21. A method of determining the localisation and the stage of pathology in an animal or human comprising the steps of:
(i) obtaining samples from different and identified parts of the animal or human, which samples are preferably selected in the group constituted by samples of epithelial tissues such as intestinal tissues, mucus samples and samples of washings collected from the tissue;
(ii) determining which of the samples collected in step (i) comprise activated I-enterocytes;
(iii) quantifying the level of activated I-enterocytes present in the samples identified in step (ii) as comprising activated I-enterocytes; and
(iv) correlating the results obtained in step (iii) to the localisation and to the stage of the pathology.

22. A method of determining the risk for a patient to develop a pathology comprising the steps of:
(i) obtaining a sample of epithelial tissue from the animal or from the human;
(ii) determining if the sample obtained in preceding step (i) comprises elevated levels of plasmalogen in the epithelial tissue;
(iii) correlating said elevated level of plasmalogen to observations made in normal non-initiated tissue; and
(iv) quantifying the stage of pathology if the animal or human has developed pathology.

23. A kit for working the method according to anyone of claims 1 to 22, comprising:
(i) at least one reagent for visualising the presence of plasmalogen (preferably the presence of I-enterocytes) in the sample(s); and
(ii) a reagent for identifying the presence of activated I-enterocytes in the sample(s).

24. A kit according to claim 23, characterised in that the reagent for visualising the presence activated I-enterocytes in the sample(s) is selected in the group constituted by mild acids and dilute mercuric chloride.

25. A kit for working the method according to anyone of claims 1 to 22, comprising:
(i) at least one reagent for oxydizing said tissue; and
(ii) a Schiff's reagent or a modified Schiff's reagent.

26. A kit for working the method according to anyone of claims 1 to 22, comprising:
(i) at least one reagent for oxidizing said tissue; and
(ii) a Schiff s reagent or a modified Schiff's reagent;and
(iii) instructions for working the method according to anyone of claims 1 to 22.

27. A kit according to claims 25 or 26, characterised in that the reagent for oxidising said tissue is selected in the group constituted by inorganic acids, by mercuric chlorides and by solutions thereof.

28. Use of a method as defined in anyone of claims 1 to 22 as a tool to investigate pathologic development such as pre-cancerous states in animal and cell culture models of disease.
